SEQUENCE LISTING (52 sequences)

SEQ ID NO: 1 - protein, 130 aa, Levivirus Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 2 - protein, 53 aa, Levivirus Bacteriophage MS2
YKVTCSVRQSSAQNRKYTIKVEVPKVATQTVGGVELPVAAWRSYLNMELTIPI

SEQ ID NO: 3 - RNA, 10 nt, Levivirus Bacteriophage MS2
  misc_feature (1)..(3): n is a, c, g, or u
  misc_feature (8)..(10): n is a, c, g, or u
nnnauuannn

SEQ ID NO: 4 - RNA, 23 nt, Levivirus Bacteriophage MS2
  misc_feature (1)..(7): n is a, c, g, or u
  misc_feature (9)..(10): n is a, c, g, or u
  misc_feature (15)..(23): n is a, c, g, or u
nnnnnnnannauuannnnnnnnn

SEQ ID NO: 5 - RNA, 23 nt, Levivirus Bacteriophage MS2
  misc_feature (1)..(7): n is a, c, g, or u
  misc_feature (9)..(10): n is a, c, g, or u
  misc_feature (15)..(23): n is a, c, g, or u
nnnnnnnannaucannnnnnnnn

SEQ ID NO: 6 - RNA, 23 nt, Levivirus Bacteriophage MS2
  misc_feature (1)..(7): n is a, c, g, or u
  misc_feature (9)..(10): n is a, c, g, or u
  misc_feature (15)..(23): n is a, c, g, or u
nnnnnnnannacuannnnnnnnn

SEQ ID NO: 7 - RNA, 23 nt, Levivirus Bacteriophage MS2
aaacaugaggauuacccaugucg

SEQ ID NO: 8 - RNA, 23 nt, Levivirus Bacteriophage MS2
aaacaugaggaucacccaugucg

SEQ ID NO: 9 - RNA, 23 nt, Levivirus Bacteriophage MS2
aaacaugaggacuacccaugucg

SEQ ID NO: 10 - protein, 107 aa, Enterobacteria phage lambda
MDAQTRRRERRAEKQAQWKAANPLLVGVSAKPVNRPILSLNRKPKSRVESALNPIDLTVLAEYHKQIESNLQRIERKNQRTWYSKPGERGITCSGRQKIKGKSIPLI

SEQ ID NO: 11 - protein, 100 aa, Enterobacteria phage P22
MTVITYGKSTFAGNAKTRRHERRRKLAIERDTICNIIDSIFGCDAPDASQEVKAKRIDRVTKAISLAGTRQKEVEGGSVLLPGVALYAAGHRKSKQITAR

SEQ ID NO: 12 - protein, 99 aa, Enterobacteria phage Phi21
MVTIVWKESKGTAKSRYKARRAELIAERRSNEALARKIALKLSGCVRADKAASLGSLRCKKAEEVERKQNRIYYSKPRSEMGVTCVGRQKIKLGSKPLI

SEQ ID NO: 13 - protein, 21 aa, Enterobacteria phage lambda
DAQTRRRERRAEKQAQWKAAN

SEQ ID NO: 14 - protein, 25 aa, Enterobacteria phage P22
KSTFAGNAKTRRHERRRKLAIERDT

SEQ ID NO: 15 - protein, 22 aa, Enterobacteria phage Phi21
ESKGTAKSRYKARRAELIAERR

SEQ ID NO: 16 - RNA, 15 nt, Enterobacteria phage lambda
gcccugaagaagggc

SEQ ID NO: 17 - RNA, 15 nt, Enterobacteria phage lambda
gcccugaaaaagggc

SEQ ID NO: 18 - RNA, 15 nt, Enterobacteria phage P22
gcgcugacaaagcgc

SEQ ID NO: 19 - RNA, 20 nt, Enterobacteria phage Phi21
uucaccucuaaccgggugag

SEQ ID NO: 20 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQDCSADDDNFLVPIAVGAALAGVLILVLLAYFIGLKHHHAGYEQF

SEQ ID NO: 21 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQECSLDDDTILIPIIVGAGLSGLIIVIVIAYVIGRRKSYAGYQTL

SEQ ID NO: 22 - protein, 411 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAEECSADSDLNFLIPVAVGVALGFLIIVVFISYMIGRRKSRTGYQSV

SEQ ID NO: 23 - protein, 10 aa, Homo sapiens
KHHHAGYEQF

SEQ ID NO: 24 - protein, 11 aa, Homo sapiens
RRKSYAGYQTL

SEQ ID NO: 25 - protein, 11 aa, Homo sapiens
RRKSRTGYQSV

SEQ ID NO: 26 - protein, 417 aa, Homo sapiens
MAAPGSARRPLLLLLLLLLLGLMHCASAAMFMVKNGNGTACIMANFSAAFSVNYDTKSGPKNMTFDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSMLIPIAVGGALAGLVLIVLIAYLVGRKRSHAGYQTI

SEQ ID NO: 27 - protein, 11 aa, Homo sapiens
RKRSHAGYQTI

SEQ ID NO: 28 - protein, 238 aa, Homo sapiens
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 29 - protein, 215 aa, Homo sapiens
MAVEGGMKCVKFLLYVLLLAFCGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 30 - protein, 166 aa, Homo sapiens
CGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 31 - protein, 14 aa, Homo sapiens
CCLVKSIRSGYEVM

SEQ ID NO: 32 - protein, 478 aa, Homo sapiens
MGRCCFYTAGTLSLLLLVTSVTLLVARVFQKAVDQSIEKKIVLRNGTEAFDSWEKPPLPVYTQFYFFNVTNPEEILRGETPRVEEVGPYTYRELRNKANIQFGDNGTTISAVSNKAYVFERDQSVGDPKIDLIRTLNIPVLTVIEWSQVHFLREIIEAMLKAYQQKLFVTHTVDELLWGYKDEILSLIHVFRPDISPYFGLFYEKNGTNDGDYVFLTGEDSYLNFTKIVEWNGKTSLDWWITDKCNMINGTDGDSFHPLITKDEVLYVFPSDFCRSVYITFSDYESVQGLPAFRYKVPAEILANTSDNAGFCIPEGNCLGSGVLNVSICKNGAPIIMSFPHFYQADERFVSAIEGMHPNQEDHETFVDINPLTGIILKAAKRFQINIYVKKLDDFVETGDIRTMVFPVMYLNESVHIDKETASRLKSMINTTLIITNIPYIIMALGVFFGLVFTWLACKGQGSMDEGTADERAPLIRT

SEQ ID NO: 33 - protein, 335 aa, Homo sapiens
MGRCCFYTAGTLSLLLLVTSVTLLVARVFQKAVDQSIEKKIVLRNGTEAFDSWEKPPLPVYTQFYFFNVTNPEEILRGETPRVEEVGPYTYRSLDWWITDKCNMINGTDGDSFHPLITKDEVLYVFPSDFCRSVYITFSDYESVQGLPAFRYKVPAEILANTSDNAGFCIPEGNCLGSGVLNVSICKNGAPIIMSFPHFYQADERFVSAIEGMHPNQEDHETFVDINPLTGIILKAAKRFQINIYVKKLDDFVETGDIRTMVFPVMYLNESVHIDKETASRLKSMINTTLIITNIPYIIMALGVFFGLVFTWLACKGQGSMDEGTADERAPLIRT

SEQ ID NO: 34 - protein, 19 aa, Homo sapiens
GQGSMDEGTADERAPLIRT

SEQ ID NO: 35 - protein, 156 aa, Homo sapiens
MITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 36 - protein, 511 aa, Vesicular Stomatitis Virus
MKCLLYLAFLFIGVNCKFTIVFPHNQKGNWKNVPSNYHYCPSSSDLNWHNDLVGTALQVKMPKSHKAIQADGWMCHASKWVTTCDFRWYGPKYITHSIRSFTPSVEQCKESIEQTKQGTWLNPGFPPQSCGYATVTDAEAAIVQVTPHHVLVDEYTGEWVDSQFINGKCSNDICPTVHNSTTWHSDYKVKGLCDSNLISMDITFFSEDGELSSLGKKGTGFRSNYFAYETGDKACKMQYCKHWGVRLPSGVWFEMANKDLFAAARFPECPEGSSISAPSQTSVDVSLIQDVERILDYSLCQETWSKIRAGLPISPVDLSYLAPKNPGTGPVFTIINGTLKYFETRYIRVDIAAPILSRMVGMISGTTKERVLWDDWAPYEDVEIGPNGVLRTSSGYKFPLYMIGHGMLDSDLHLSSKAQVFEHPHIQDAASQLPDGETLFFGDTGLSKNPIEFVEGWFSSWKSSIASFFFTIGLIIGLFLVLRVGIYLCIKLKHTKKRQIYTDIEMNRLGK

SEQ ID NO: 37 - protein, 3 aa, Artificial
  Consensus amino acid sequence of sequon for N-linked glycosylation
NST

SEQ ID NO: 38 - protein, 5 aa, Artificial
  Consensus sequence for sequon for N-linked glycosylation
GNSTM

SEQ ID NO: 39 - protein, 8 aa, Artificial
  FLAG epitope for tagging proteins
DYKDDDDK

SEQ ID NO: 40 - protein, 21 aa, Homo sapiens
AAVLVLLVIVIISLIVLVVIW

SEQ ID NO: 41 - protein, 10 aa, Artificial
  Glycine-serine rich linking sequence for VL and VH domains in scFv
GLGSGSGGSS

SEQ ID NO: 42 - protein, 10 aa, Artificial
  Glycine-serine rich linking sequence between VL and VH domains of scFv
GSGSGSGGSS

SEQ ID NO: 43 - protein, 15 aa, Artificial
  Glycine-serine rich linking sequence between VL and VH domains of scFv
GGGGSGGGGSGGGGS

SEQ ID NO: 44 - protein, 40 aa, Artificial
  Glycine-serine rich linking sequence betweent VL and VH domains of scFv
SGGGSGGGSGGGSGGSGGSGGGSGGSGGSGGGSGGGSGGG

SEQ ID NO: 45 - protein, 23 aa, Artificial
  Helical linking sequence for linking domains of fusion protein
DQSNSEEAKKEEAKKEEAKKSNS

SEQ ID NO: 46 - protein, 12 aa, Artificial
  Hinge sequence of IgG4 for linking domains of fusion protein
ESKYGPPAPPAP

SEQ ID NO: 47 - protein, 27 aa, Artificial Sequence
  synthetic
TGDQSNSEEAKKEEAKKEEAKKSNSID

SEQ ID NO: 48 - protein, 16 aa, Artificial Sequence
  synthetic
TGESKYGPPAPPAPID

SEQ ID NO: 49 - protein, 44 aa, Artificial Sequence
  synthetic
TGSGGGSGGGSGGGSGGSGGSGGGSGGSGGSGGGSGGGSGGGID

SEQ ID NO: 50 - protein, 14 aa, Artificial Sequence
  synthetic
TGGLGSGSGGSSID

SEQ ID NO: 51 - protein, 14 aa, Artificial Sequence
  synthetic
TGGSGSGSGGSSID

SEQ ID NO: 52 - protein, 19 aa, Artificial Sequence
  synthetic
TGGGGGSGGGGSGGGGSID

(19) United States
(12) Patent Application Publication
(10) Pub. No.: US 2021/0030850 A1
(43) Pub. Date: Feb. 4, 2021

(54) EXTRACELLULAR VESICLES COMPRISING TARGETING AFFINITY DOMAIN-BASED MEMBRANE PROTEINS

(71) Applicant: Northwestern University, Evanston, IL (US)

(72) Inventors: Joshua N. LEONARD, Wilmette, IL (US); Devin M. STRANFORD, Evanston, IL (US)

(73) Assignee: Northwestern University, Evanston, IL (US)

(21) Appl. No.: 17/046,147

(22) PCT Filed: Apr. 10, 2019
(86) PCT No.: PCT/US2019/026751

§ 371 Date: Oct. 8, 2020

Related U.S. Application Data
(60) Provisional application No. 62/655,521, filed on Apr. 10, 2018.

(51) Int. Cl. A61K 38/46; A61K 9/00; A61K 9/127; C07K 16/28

(52) U.S. Cl. CPC A61K 38/465; A61K 9/0019; C07K 2319/03; C07K 16/2806; C07K 2317/622; A61K 9/1272

(57) ABSTRACT

Disclosed are extracellular vesicles comprising an engineered targeting protein for targeting the extracellular vesicles to target cells. The targeting protein is a fusion protein that includes (i) an affinity agent, such as a single-chain variable fragment of an antibody (scFv), which is expressed on the surface of the extracellular vesicles and (ii) a transmembrane domain, and may include additional domains. Exemplary extracellular vesicles may include but are not limited to exosomes or microvesicles.

### CROSS-REFERENCE TO RELATED PATENT APPLICATIONS

The present application claims the benefit of priority under 35 U.S.C. § 119(e) to U.S. Provisional Application No. 62/655,521, filed on Apr. 10, 2018, the contents of which are incorporated herein by reference in their entireties.

### STATEMENT REGARDING FEDERALLY SPONSORED RESEARCH OR DEVELOPMENT

This invention was made with government support under P30AI117943 awarded by the National Institutes of Health. The government has certain rights in the invention.

### BACKGROUND

The field of the invention relates to the use of lipid particles for delivering agents to target cells. In particular, the field of the invention relates to secreted extracellular vesicles (EVs) that contain a targeting affinity domain based membrane protein such as a single chain antibody domain. The secreted extracellular vesicles may be utilized to deliver an agent to a target cell, such as a therapeutic agent.

Secreted extracellular vesicles, such as exosomes and microvesicles, are nanometer-scale lipid vesicles that are produced by many cell types and transfer proteins, nucleic acids, and other molecules between cells in the human body, as well as those of other animals. Targeted exosomes in particular have a wide variety of potential therapeutic uses and have already been shown to be effective for delivery of RNA to neural cells and tumor cells in mice.

Here, we describe a method for displaying targeting affinity domain-based membrane proteins on the surface of exosomes and microvesicles through exosome and microsome biogenesis, respectively. The disclosed technology utilizes affinity agents, such as antibodies or antigen-binding domains of antibodies, to provide affinity domains for the targeting membrane proteins. In particular, the described technology provides a robust method for display of targeting proteins on the surface of EVs via the expression of engineered proteins that localize to EVs and exhibit external affinity domains. The disclosed targeting system can be used for engineering EVs for use in targeted gene therapy or targeted drug delivery vehicles in vivo. As such, the disclosed technology may be used for engineering targeted EVs which could be applied to a wide variety of cell types and diseases.

### SUMMARY

Disclosed are extracellular vesicles comprising an engineered targeting protein that targets the extracellular vesicles to a target cell, tissue, or pathway. The engineered targeting protein may target the extracellular vesicles to a target cell by targeting a surface protein of the target cell endocytosis via specific routes. The targeting protein is a fusion protein that minimally includes as domains, (i) an affinity agent, such as a single-chain variable fragment of an antibody (scFv), wherein the scFv is expressed on the surface of the extracellular vesicles; and (ii) a transmembrane domain that orients the fusion protein in the membrane of the extracellular vesicles. Exemplary extracellular vesicles may include but are not limited to exosomes and microvesicles.

The engineered targeting proteins or “fusion proteins” of the extracellular vesicles further may include additional domains. Additional domains may include engineered glycosylation sites, for example, which enable the fusion protein to be glycosylated in the cell. Preferably, when the engineered glycosylation site is glycosylated, the fusion protein and/or the component domains of the fusion protein are protected from cleavage of the fusion protein and/or degradation in lysosomes. For example, when the engineered glycosylation site is glycosylated, preferably the scFv is protected from being cleaved from the remainder of the fusion protein.

Additional domains of the fusion proteins may include exosome-targeting domains. Preferably, the exosome-targeting domains target the fusion proteins to intracellular vesicles such as lysosomes, where the fusion proteins may be incorporated into the membranes of lysosomes and secreted in extracellular vesicles such as exosomes.

Additional domains of the fusion proteins may include microvesicle-targeting domains. Preferably, the microvesicle-targeting domains target the fusion proteins to the cell surface, where the fusion proteins may be incorporated into the cell membranes and secreted in extracellular vesicles such as microvesicles.

The extracellular vesicles further may comprise an agent, such as a therapeutic agent, and the extracellular vesicles may be utilized to deliver the comprised agent to a target cell. Agents comprised by the extracellular vesicles may include but are not limited to biological molecules, such as cargo RNAs, and other small molecular therapeutic molecules or proteins. For example, the fusion protein further may comprise an RNA-binding domain that binds to one or more RNA-motifs present on a cargo RNA such that the fusion protein functions as a packaging protein in order to package the cargo RNA into the extracellular vesicle, prior to the extracellular vesicles being secreted from a cell. In some embodiments, the packaging protein may be referred to as an extracellular vesicle-loading protein or an “EV-loading protein.”

### BRIEF DESCRIPTION OF THE FIGURES

FIG. 1. Overview of combinatorial sgRNA therapy to cure HIV infection.

FIG. 2. Suppression of viral replication in Cas9-expressing SupT1 cells receiving combinatorial sgRNAs. (See Wang et al. “A Combinatorial CRISPR-Cas9 Attack on HIV-1 DNA Extinguishes All Infectious Provirus in Infected T Cell Cultures, Cell Reports, Volume 17, Issue 11, p2819-2826, Dec. 13, 2016; the content of which is incorporated herein by reference in its entirety).

FIG. 3. Overview of EV production and EV-mediated biomolecule delivery. (See Stranford and Leonard, “Delivery of Biomolecules via Extracellular Vesicles: A Budding Therapeutic Strategy, Advances in Genetics, 98:155-175, Sep. 11, 2017; the content of which is incorporated herein by reference in its entirety). Production: Exosomes are formed by the invagination of endosomal membranes to form multivesicular bodies (MVBs), and back-fusion of MVBs with the plasma membrane releases exosomes from the cell. Microvesicles are formed by direct budding from the plasma membrane. Both types of vesicle incorporate RNA and protein from the producer cell, but exosomes are enriched in endosomal membrane proteins. Uptake: EVs can be taken up by a variety of endocytic routes by recipient cells or by direct fusion at the cell surface. Cargo delivery: Release of EV cargo into the cytoplasm of a recipient cell requires fusion between EV and cellular membranes in either endosomal compartments or at the plasma membrane. Failure to fuse results in degradation of EVs and their cargo via the endosomal-lysosomal pathway.

FIG. 4. Schematic of EV-mediated Cas9 and combinatorial sgRNA delivery to T cells and Cas9-mediated cleavage of the HIV provirus in latently infected T cells.

FIG. 5. Schematic of EVs displaying anti-CD2 scFv which target the EVs to CD2-bearing cells such as latently infected T cells.

FIG. 6. Schematic of EVs displaying measles virus glycoprotein variants H and F which target the EVs to CD46-bearing cells and Signaling Lymphocyte Activation Molecule (SLAM)-bearing cells (SLAM-bearing).

FIG. 7. Schematic of EVs displaying Intercellular Adhesion Molecule 1 (ICAM-1) which targets the EVs to Lymphocyte Function-Associated Antigen 1 (LFA-1)-bearing cells, such as activated T cells.

FIG. 8. Method of loading EVs with Cas9 and sgRNA.

FIG. 9. Anti-CD2 scFv localization to EVs (N terminal detection). HEK293FT cells were transfected with constructs encoding either the FLAG-tagged CD2 scFv fused to the PDGFR transmembrane domain or a FLAG tag fused to the PDGFR transmembrane domain as an EV-display control. Cell lysates (2 μg) or EVs (8.9×10⁸ per lane) were loaded and constructs were detected by anti-FLAG antibodies (FLAG tags are located at the N terminus of all display constructs). The positive signal in lanes 9 and 10 indicate that the N terminus of the protein (which includes the scFv domain on the EV surface) is detected for both microvesicles and exosomes.

FIG. 10. Anti-CD2 scFv localization to EVs (C terminal detection). HEK293FT cells were transfected with constructs encoding either the FLAG-tagged CD2 scFv fused to the PDGFR transmembrane domain or a FLAG tag fused to the PDGFR transmembrane domain as an EV-display control. Cell lysates (2 μg) or EVs (8.9×10⁸ per lane) were loaded and constructs were detected by anti-HA antibodies (HA tags located at the C terminus). The positive signal in lanes 9 and 10 indicate that the C terminus of the protein (which includes the intracellular HA tag) is detected for both microvesicles and exosomes.

FIG. 11. Schematic of Cas9-loaded EVs and sgRNA-loading EVs and functional delivery to recipient T cells.

### DETAILED DESCRIPTION

The present invention is described herein using several definitions, as set forth below and throughout the application.

Unless otherwise specified or indicated by context, the terms “a”, “an”, and “the” mean “one or more.” For example, “a fusion protein,” “an RNA,” and “a loop” should be interpreted to mean “one or more fusion proteins,” “one or more RNAs,” and “one or more loops,” respectively. An “engineered glycosylation site” should be interpreted to mean “one or more engineered glycosylation sites.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

Disclosed are extracellular vesicles comprising a targeting protein that targets the extracellular vesicles to a target cell. Exemplary extracellular vesicles may include but are not limited to exosomes. However, the term “extracellular vesicles” should be interpreted to include all nanometer-scale lipid vesicles that are secreted by cells such as secreted vesicles formed from lysosomes or vesicles secreted by budding from the plasma membrane or by other cellular membrane budding processes.

The disclosed extracellular vesicles comprise a “targeting protein.” The target protein may be described as a “fusion protein,” and the term “targeting protein” and “fusion protein” may be used interchangeably herein depending on context. The fusion protein typically includes: (i) affinity agent, such as a single chain variable fragment of an antibody (scFv), that is expressed on the surface of the extracellular vesicles and preferably targets the extracellular vesicles to target cells and (ii) a transmembrane domain, which preferably orients the fusion protein in the membrane of the extracellular vesicles. In some embodiments, the fusion protein has a luminal or extracellular N-terminal end and a cytosolic C-terminal end.

By “affinity agent” we mean to include moieties that will facilitate specific binding of the EV to a target cell. Preferred moieties are protein domains (preferably folded protein domains] and are not unfolded peptides. Sample affinity agents include (but are not limited to) scFv, camelid nanobodies, fibronectin domain-derived monobodies, and DARPins (see Koide A, Koide S, 2007; Nanobodies: antibody mimics based on the scaffold of the fibronectin type III domain, Methods Mol Biol 352: 95-109; Nanobodies: Natural Single-Domain Antibodies, Annual Review of Biochemistry, Vol 82: 775-797, 2013; Designed Ankyrin Repeat Proteins (DARPins): Binding Proteins for Research, Diagnostic, and Therapy, Ann Rev of Pharm Tox, Vol 55:489-511, 2015).

The fusion protein of the disclosed extracellular vesicles typically includes a single chain antibody such as a scFv. Single chain antibodies may be formed by linking a heavy chain variable domain fragment and a light chain variable domain fragment (Fv region) via an amino acid linker, resulting in a single polypeptide chain. Such single-chain Fvs or “scFv's” have been prepared by fusing DNA encoding a peptide linker between DNAs encoding the two variable domain polypeptides (V_{L} and V_{H}). The carboxy terminal end of the V_{L} fragment may be fused in frame via a linker to the amino terminal end of the V_{H} fragment, or vice versa, where the carboxy terminal end of the V_{H} fragment may be fused in frame via a linker to the amino terminal end of the V_{L} fragment. The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108). The linker is usually 10-50 amino acids in length and is rich in glycine for flexibility, as well as serine or threonine for solubility, and can either connect the N-terminus of the V_{L} with the C-terminus of the V_{H}, or vice versa. Because the linker between the V_{L} and the V_{H} domains may be rich in glycine and serine (and/or threonine), the linker between the V_{L} and the V_{H} domains is sometimes referred to as a “GS” linker. Suitable GS linkers may include, but are not limited to: GS linkers having 10 amino acids such as GLGSGSGGSS (SEQ ID NO:41) or GSGSGSGGSS (SEQ ID NO:42); GS linkers having 15 amino acids such as GGGGSGGGGSGGGGS (SEQ ID NO:43); and GS linkers having 40 amino acids such as SGGGSGGGSGGGSGGSGGSGGGSGGSGGSGGGSGGGSGGG (SEQ ID NO:44). The linker between the V_{L} and the V_{H} domains may be referred to herein as a L₁ linker, which is distinguished from the L₂ linker discussed below.

By combining and linking different V_{L}'s and V_{H}'s, multimeric scFvs that bind to different epitopes can be formed such as diabodies, tribodies, and tetrabodies. (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol. Biol. 178:379-87; the contents of which are incorporated herein by reference in their entireties. The multimeric scFvs may be monospecific (i.e., specific for a single epitope) or multi-specific (i.e., having specific for two or more epitopes).

The affinity agent, such as a scFv, of the fusion protein typically binds to an epitope present on the surface of a target cell. The scFv of the fusion protein typically is present at the luminal end of the fusion protein, which optionally may be the N-terminus of the fusion protein. For example, the fusion protein may comprise a structure as follows: Nₜₑᵣ-signal peptide-scFV-transmembrane domain-Cₜₑᵣ.

The fusion protein of the disclosed extracellular vesicles typically includes a transmembrane domain. Transmembrane domains are known in the art. Transmembrane domains (TMDs) consist predominantly of nonpolar amino acid residues and may traverse the bilayer once (single pass) or several times. TMDs usually consist of a helices. The peptide bond is polar and can include internal hydrogen bonds formed between carbonyl oxygen atoms and amide nitrogen atoms which may be hydrated. Within the lipid bilayer, where water is essentially excluded, peptides usually adopt the α-helical configuration in order to maximize their internal hydrogen bonding. A length of helix of 18-21 amino acid residues is usually sufficient to span the usual width of a lipid bilayer. TMDs that are oriented with an extracytoplasmic N-terminus and a cytoplasmic C-terminus are classified as type I TMDs, and TMDs that are oriented with an extracytoplasmic C-terminus and a cytoplasmic N-terminus are classified as type II TMDs. In some embodiments of the disclosed e extracytoplasmic, they are classified as type I or, if cytoplasmic, type II. In some embodiments, the fusion protein of the disclosed extracellular vesicles is a single pass, type I transmembrane domain comprising 18-21 amino acids, where at least about 90% of the amino acids are nonpolar. Suitable TMDs for the disclosed fusion proteins may include the transmembrane domain of cellular receptors, such as the platelet-derived growth factor receptor (PDGFR), which sequence is provided as SEQ ID NO:40. The TMD may be linked directly to the affinity agent (such as ascFv) or the TMD may be linked via a linker referred to herein as L₂. (i.e., where the fusion protein comprises a linker between V_{L} and V_{H} (L₁) and a linker between V_{H} and TMD (L₂)). Suitable linking sequences for L₂ may include amino acid sequences comprising about 10-50 amino acids selected from glycine, serine (and/or threonine) (e.g., so-called GS linkers) or other linking sequences such as helical linkers and hinge linkers present in immunoglobulins. Suitable GS linkers may include, but are not limited to: GS linkers having 10 amino acids such as GLGSGSGGSS (SEQ ID NO:41) or GSGSGSGGSS (SEQ ID NO:42); GS linkers having 15 amino acids such as GGGGSGGGGSGGGGS (SEQ ID NO:43); and GS linkers having 40 amino acids such as SGGGSGGGSGGGSGGSGGSGGGSGGSGGSGGGSGGGSGGG (SEQ ID NO:44). Suitable helical linkers may include but are not limited to DQSNSEEAKKEEAKKEEAKKSNS (SEQ ID NO:45). Suitable hinge linkers may include the hinge linker of IgG4 having an amino acid sequence ESKYGPPAPPAP (SEQ ID NO:46). Other suitable linkers may have flanking sequences originating from restriction sites, such as helical linker: TGDQSNSEEAKKEEAKKEEAKKSNSID (SEQ ID NO: 47); IgG4 hinge linker: TGESKYGPPAPPAPID (SEQ ID NO: 48); 40 GS linker: TGSGGGSGGGSGGGSGGSGGSGGGSGGSGGSGGGSGGGSGGGID (SEQ ID NO: 49); 10 GS linker: TGGLGSGSGGSSID or TGGSGSGSGGSSID (SEQ ID NO: 50 and 51); 15 GS linker: TGGGGGSGGGGSGGGGSID (SEQ ID NO: 52).

The fusion protein of the disclosed extracellular vesicles may optionally include an engineered tag that can be utilized to detect or isolate the fusion protein. For example, the fusion protein may include an artificial epitope at its N-terminus, C-terminus, or both, such as a FLAG epitope (SEQ ID NO:39). Other suitable engineered tags may include histidine tags comprising 4-10 histidine residues, or a hemagglutinin (HA) tag comprising 9 amino acids.

The fusion protein of the disclosed extracellular vesicles may optionally include an engineered glycosylation site (EGS) (e.g., a heterologous glycosylation site that is not naturally occurring in any of the amino acids sequence of the domains of the fusion protein). The engineered glycosylation site of the fusion protein may be defined as a sequence of amino acids that is a target for enzymatic, N-linked glycosylation when the fusion protein is expressed in a cell. The engineered glycosylation site may be present adjacent to the scFv of the fusion protein (e.g., Nₜₑᵣ-signal peptide-scFv-engineered glycosylation site (EGS)-TMD-Cₜₑᵣ). Preferably, when the engineered glycosylation site is glycosylated, the fusion protein or the component domains of the fusion protein are protected from cleavage from the fusion protein and/or degradation in lysosomes. (See Hung et al.; and Schulz). For example, the fusion protein may include a glycosylation motif and/or may be engineered to include a glycosylation motif in order to protect or inhibit the fusion protein and/or component domains of the fusion protein from proteolytic cleavage from the fusion protein or degradation, such as intracellular proteolysis. (See Kundra et al.). Suitable glycosylation motifs may include the NX(S/T) consensus sequon and in particular the NST sequon (SEQ ID NO:37). In some embodiments, the fusion protein may include a GNSTM sequon (SEQ ID NO:38). The NST sequence is a known N-linked glycosylation sequon, and the amino acids G and M flanking the sequon may increase glycosylation frequency in mammals. (See Baño-Polo et al.). The glycosylation site typically is “engineered,” meaning that the glycosylation site typically is not naturally present in the fusion protein or any of the component proteins of the fusion protein, and rather, is introduced into the fusion protein, for example, by recombinant engineering.

The fusion protein of the disclosed extracellular vesicles may optionally include an exosome-targeting domain (ETD). The exosome targeting domain of the fusion protein may include but is not limited to a domain of an exosomal-associated protein and/or a lysosome-associated protein. A database of exosomal proteins, RNA, and lipids is provided by ExoCarta at its website. (See also, Mathivanan et al., Nucl. Acids Res. 2012, Vol. 40, Database issue D1241-1244, published online 11 Oct. 2011, the content of which is incorporated herein by reference in its entirety.) Suitable exosome-associated proteins, which also may be described as exosomal vesicle-enriched proteins or (EEPs) have been described. (See Hung and Leonard, “A platform for actively loading cargo RNA to elucidate limiting steps in EV-mediated delivery,” J. Extracellular Vesicles, 2016, 5: 31027, published 13 May 2016, the content of which is incorporated herein by reference in its entirety). In some embodiments, suitable domains of lysosome-associated proteins may include domains from lysosome membrane proteins having a luminal N-terminus and a cytoplasmic C-terminus, although membrane proteins having different orientations also may be suitable (e.g. membrane proteins having a luminal C-terminus and a cytoplasmic N-terminus).

The fusion protein of the disclosed extracellular vesicles may optionally include a microvesicle targeting domain. The microvesicle targeting domain may target a fusion protein to the cell surface, where the fusion protein may be incorporated into the cell membranes and secreted as extracellular vesicles such as microvesicles. Microvesicle targeting domains may include domains of cell surface proteins including domains of cell surface receptors such as G-protein coupled receptors (GCRs) including platelet-derived growth factor receptor (PDGFR). In some embodiments, a “microvesicle targeting domain” as contemplated herein is a “cell-surface targeting domain.” Cell-surface targeting domains are known in the art.

In some embodiments of the fusion proteins disclosed herein, the fusion protein includes an exosome-targeting domain and the exosome-targeting domain is an exosome-targeting domain of a LAMP. Suitable LAMPs may include, but are not limited to, LAMP-1 and LAMP-2, and isoforms thereof (See Fukuda et al., “Cloning of cDNAs Encoding Human Lysosomal Membrane Glycoproteins, h-lamp-1 and h-lamp-2,” J. Biol. Chem., Vol. 263, No. 35 Dec. 1988, pp. 18920-18928; and Fukuda, “Lysosomal Membrane Glycoproteins,” J. Biol. Chem., Vol. 266, No. 32, November 1991, pp. 21327, 21330.) LAMPs are lysosome-membrane proteins having a luminal (i.e., extracytoplasmic) N-terminus and a cytoplasmic C-terminus. (See id.). The mRNAs for expressing LAMPs may be processed differently to give isoforms. For example, there are three isoforms for LAMP-2 designated as LAMP-2a, LAMP-2b, and LAMP-2c. (See UniProt Database, entry number P13473—LAMP2_HUMAN, the contents of which is incorporated herein by reference in its entirety). LAMP-1 has a single isoform. (See UniProt Database, entry number P11279—LAMP1_HUMAN, the content of which is incorporated herein by reference in its entirety). The full-length amino acid sequence of LAMP-2a, LAMP-2b, and LAMP-2c are provided herein as SEQ ID NOs:20, 21, and 22, respectively. The full-length amino acid sequence of LAMP-1 is provided herein as SEQ ID NO:26. The fusion proteins disclosed herein may include the full-length amino acid sequence of a LAMP or a variant thereof as contemplated herein having a percentage of sequence identity in comparison to the amino acid sequence of the wild-type LAMP, or a fragment thereof comprising a portion of the wild-type LAMP (e.g., SEQ ID NOs:23, 24, 25, and 27 comprising a portion of the C-termini of LAMP-2a, LAMP-2b, LAMP-2c, and LAMP-1, respectively).

For LAMPs, the C-terminus (e.g., comprising the 10-11 C-terminal amino acids) has been shown to be important for targeting LAMPs to lysosomes. (See id.; and Fukuda 1991). In some embodiments of the disclosed extracellular vesicles, the fusion protein comprises the RNA-binding domain fused to the C-terminus of one of SEQ ID NOs:23, 24, 25, and 27, which comprise a portion of the C-termini of LAMP-2a, LAMP-2b, LAMP-2c, and LAMP-1, respectively). The fusion protein may include the cytoplasmic domain of a LAMP and optionally may include additional amino acid sequences (e.g., at least a portion of the transmembrane domain and/or at least a portion of the luminal domain).

In some embodiments, the exosome-targeting domain is an exosome-targeting domain of a LIMP. Suitable LIMPs may include, but are not limited to, LIMP-1 (CD63) and LAMP-2, and isoforms thereof. LIMPs are lysosome-membrane proteins having one or more luminal domains, multiple transmembrane domains, and a cytoplasmic C-terminus. (See Ogata et al., “Lysosomal Targeting of Limp II Membrane Glycoprotein Requires a Novel Leu-Ile Motif at a Particular Position in Its Cytoplasmic Tail,” J. Biol. Chem., Vol. 269, No. 7, February 1994, pp. 5210-5217). The mRNAs for expressing LIMPs may be processed differently to give isoforms. For example, there are three isoforms for LIMP-1 designated as LIMP-1a, LIMP-1b, and LIMP-1c and two isoforms for LIMP-2 designated as LIMP-2a and LIMP-2b. (See UniProt Database, entry number Q10148—SCRB2_HUMAN, and UniProt Database, entry number P08962—CD63_HUMAN, the content of which is incorporated herein by reference in its entirety). The full-length amino acid sequence of LIMP-1a, LIMP-1b, and LIMP-1c are provided herein as SEQ ID NOs:28, 29, and 30, respectively. The full-length amino acid sequence of LIMP-2A and LIMP-2b are provided herein as SEQ ID NOs:32 and 33, respectively. The fusion proteins disclosed herein may include the full-length amino acid sequence of a LIMP or a variant thereof as contemplated herein having a percentage of sequence identity in comparison to the amino acid sequence of the wild-type LIMP, or a fragment thereof comprising a portion of the wild-type LIMP (e.g., SEQ ID NO:31 comprising a portion of the C-termini of LIMP-1a, LIMP-1b, LIMP-1C and SEQ ID NO:34 comprising a portion of the C-termini of LIMP-2a and LIMP-2b).

For LIMPs, the C-terminus (e.g., comprising the 14-19 C-terminal amino acids) has been shown to be important for targeting LAMPs to lysosomes. (See Ogata et al.). In some embodiments of the disclosed extracellular vesicles, the fusion protein comprises the RNA-binding domain fused to the C-terminus of one of SEQ ID NOs:31 and 34, which comprise a portion of the C-termini of LIMP-1a, LIMP-1b, LIMP-1c, and LIMP-2a and LIMP-2b). The fusion protein may include the cytoplasmic domain of a LIMP and optionally may include additional amino acid sequences (e.g., at least a portion of the transmembrane domain and/or at least a portion of the luminal domain).

In some embodiments of the fusion proteins disclosed herein the exosome-targeting domain is an exosome-targeting domain of CD63 or isoforms thereof. The CD63 protein alternately may be referred to by aliases including Lysosome-Integrated Membrane Protein 1 (LIMP-1), MLA1, Lysosomal-Associated Membrane Protein 3, Ocular Melanoma-Associated Antigen, Melanoma 1 Antigen, Melanoma-Associated Antigen ME491, Tetraspanin-30, Granulophysin, and Tspan-30. Isoforms of CD63 may include CD63 Isoform A (i.e., LIMP-1a (SEQ ID NO:28)), CD63 Isoform C (i.e., LIMP-1b (SEQ ID NO:29)) and CD63 Isoform D Precursor (provided herein as SEQ ID NO:35).

In some embodiments of the fusion proteins disclosed herein the exosome-targeting domain is an exosome-targeting domain of a viral transmembrane protein. Viral transmembrane proteins are known in the art. (See e.g., Fields Virology, Sixth Edition, 2013. See also White et al., Crit. Rev. Biochem. Mol. Biol. 2008; 43(3): 189-219). Specifically, the exosome-targeting domain may be an exosome-targeting domain of the G glycoprotein of Vesicular Stomatitis Virus (VSV G-protein). The amino acid sequence of VSV G-protein is provided herein as SEQ ID NO:36.

The disclosed extracellular vesicles further may comprise an agent, such as a therapeutic agent, where the extracellular vesicles deliver the agent to a target cell. Agents comprised by the extracellular vesicles may include but are not limited to therapeutic drugs (e.g., small molecule drugs), therapeutic proteins, and therapeutic nucleic acids (e.g., therapeutic RNA). In some embodiments, the disclosed extracellular vesicles comprise a therapeutic RNA as a so-called “cargo RNA.” For example, in some embodiments the fusion protein further may comprise an RNA-domain (e.g., at a cytosolic C-terminus of the fusion protein) that binds to one or more RNA-motifs present in the cargo RNA in order to package the cargo RNA into the extracellular vesicle, prior to the extracellular vesicles being secreted from a cell. As such, the fusion protein may function as both of a “targeting protein” and a “packaging protein.” In some embodiments, the packaging protein may be referred to as extracellular vesicle-loading protein or “EV-loading protein.” (See Hung and Leonard, “A platform for actively loading cargo RNA to elucidate limiting steps in EV-mediated delivery,” J. Extracellular Vesicles, 2016, 5: 31027, published 13 May 2016, the content of which is incorporated herein by reference in its entirety.)

In summary, the fusion protein of the disclosed extracellular vesicles in some embodiments may have a structure characterized as Nₜₑᵣ-signal peptide-(optional tag)-V_{L}-L₁-V_{H}-(optional one or more EGS and/or optional one or more linkers L₂ in any order)-TMD-(optional ETD)-(optional RBD)-(optional tag)-Cₜₑᵣ or Nₜₑᵣ-signal peptide-(optional tag)-V_{L}-L₁-V_{H} (optional one or more EGS and/or optional one or more linkers L₂ in any order)-TMD-(optional ETD)-(optional RBD)-(optional tag)-Cₜₑᵣ, where Nₜₑᵣ is the N-terminus, V_{L} is a variable light chain fragment of an antibody, L₁ is a linker of about 10-50 amino acids selected from glycine, serine, and threonine (e.g., SEQ ID NOs:41, 42, 43, or 44), V_{H} is a variable heavy chain fragment of an antibody, EGS is an optionally engineered glycosylation site, L₂ is a linker of about 10-50 amino acids (e.g., SEQ ID NOs:41, 42, 43, 44, 45, or 46), TMD is a transmembrane domain, ETD is an optional exosome-targeting domain, RBD is an optional RNA-binding domain, and Cₜₑᵣ is the C-terminus.

The disclosed extracellular vesicles may include a cargo nucleic acid such as a cargo RNA. In embodiments in which the extracellular vesicles comprise a cargo RNA, the cargo RNA which may be described as a fusion RNA comprising: (1) a RNA-motif that binds the RNA-binding domain of the fusion protein and further, (2) additional functional RNA sequences that be utilized for therapeutic purposes (e.g., miRNA, shRNA, mRNA, ncRNA, sgRNA or a combination of any of these RNAs). The RNA may also be passively loaded.

The cargo RNA of the disclosed extracellular vesicles may be of any suitable length. For example, in some embodiments the cargo RNA may have a nucleotide length of at least about 10 nt, 20 nt, 30 nt, 40 nt, 50 nt, 100 nt, 200 nt, 500 nt, 1000 nt, 2000 nt, 5000 nt, or longer. In other embodiments, the cargo RNA may have a nucleotide length of no more than about 5000 nt, 2000 nt, 1000 nt, 500 nt, 200 nt, 100 nt, 50 nt, 40 nt, 30 nt, 20 nt, or 10 nt. In even further embodiments, the cargo RNA may have a nucleotide length within a range bounded by any of these contemplated nucleotide lengths, for example, a nucleotide length between a range of about 10 nt-5000 nt, or other ranges. The cargo RNA of the disclosed extracellular vesicles may be relatively long, for example, where the cargo RNA comprises an mRNA or another relatively long RNA.

Suitable RNA-binding domains and RNA-motifs for the components of the presently disclosed extracellular vesicles may include, but are not limited to, RNA-binding domains and RNA-motifs of bacteriophage. (See, e.g., Keryer-Bibens et al., “Tethering of proteins to RNAs by bacteriophage proteins,” Biol. Cell (2008) 100, 125-138, the content of which is incorporated herein by reference in its entirety).

In some embodiments of the disclosed extracellular vesicles, the RNA-binding domain of the fusion protein is an RNA-binding domain of coat protein of MS2 bacteriophage or R17 bacteriophage, which may be considered to be interchangeable. (See, e.g., Keryer-Bibens et al.; and Stockley et al., “Probing sequence-specific RNA recognition by the bacteriophage MS2 coat protein,” Nucl. Acids. Res., 1995, Vol. 23, No. 13, pages 2512-2518, the content of which is incorporated herein by reference in its entirety). The full-length amino acid sequence of the coat protein of MS2 bacteriophage is provided herein as SEQ ID NO:1. The fusion proteins disclosed herein may include the full-length amino acid sequence of the coat protein of MS2 bacteriophage or a variant thereof as contemplated herein having a percentage of sequence identity in comparison to the amino acid sequence of the coat protein of MS2 bacteriophage, or a fragment thereof comprising a portion of the coat protein of MS2 bacteriophage (e.g., the RNA-binding domain of MS2 or SEQ ID NO:2, comprising the amino acid sequence (2-22) of the coat protein of MS2 bacteriophage).

In embodiments where the fusion protein comprises an RNA-binding domain of coat protein of MS2 bacteriophage, the cargo RNA typically comprises an RNA-motif of MS2 bacteriophage RNA which may form a high affinity binding loop that binds to the RNA-binding domain of the fusion protein. (See Peabody et al., “The RNA binding site of bacteriophage MS2 coat protein,” The EMBO J., vol. 12, no. 2, pp. 595-600, 1993; Keryer-Bibens et al.; and Stockley et al., the contents of which are incorporated herein by reference in their entireties). The RNA-motif of MS2 bacteriophage and R17 bacteriophage has been characterized. (See id.). The RNA-motif has been determined to comprise minimally a 21-nt stem-loop structure where the identity of the nucleotides forming the stem do not appear to influence the affinity of the coat protein for the RNA-motif, but where the sequence of the loop contains a 4-nt sequence (AUUA (SEQ ID NO:3)), which does influence the affinity of the coat protein for the RNA-motif. Also important, is an unpaired adenosine two nucleotides upstream of the loop. In some embodiments of the disclosed extracellular vesicles, the RNA-motif comprises one or more wild-type and/or high affinity binding loops comprising a sequence and structure selected from the group consisting of:

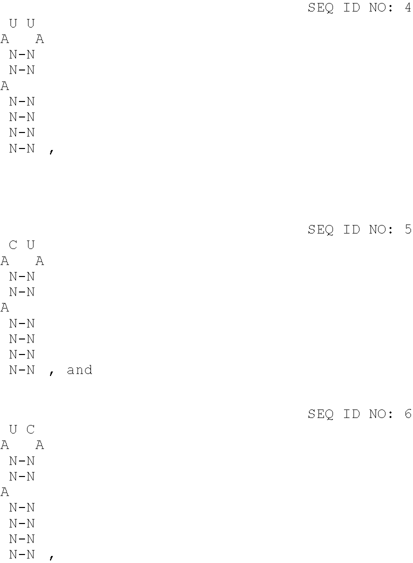

where N—N is any two base-paired RNA nucleotides (e.g., where each occurrence of N—N is independently selected from any of A-U, C-G, G-C, G-U, U-A, or U-G, and each occurrence of N—N may be the same or different). Specifically, the high affinity binding loop may comprise a sequence selected from the group consisting of SEQ ID NO:7 (5′-ACAUGAGGAUUACCCAUGU-3′), SEQ ID NO:8 (5′-ACAUGAGGACUACCCAUGU-3′), and SEQ ID NO:9 (5′-ACAUGAGGAUCACCCAUGU-3′), or a variant thereof having a percentage sequence identity.

Preferably, the RNA-binding domain of the fusion protein binds to the RNA-motif with an affinity of at least about 1×10⁻⁸ M. More preferably, the RNA-binding domain of the fusion protein binds to the RNA-motif with an affinity of at least about 1×10⁻⁹ M, even more preferably with an affinity of at least about 1×10⁻¹⁰ M.

In addition to the RNA-motif for binding to the RNA-binding domain of the fusion protein, the cargo RNA may include additional functional RNA sequences that be utilized for therapeutic purposes (e.g., miRNA, shRNA, mRNA, ncRNA, sgRNA, or a combination of any of these RNAs). (See Marcus et al., “FedExosomes: Engineering Therapeutic Biological Nanoparticles that Truly Deliver,” Pharmaceuticals 2013, 6, 659-680; Gyorgy et al., Therapeutic application of extracellular vesicles: clinical promise and open questions,” Annu. Rev. Pharmacol. Toxicol. 2015; 55:439-64, Epub 2014 Oct. 3, the contents of which are incorporated herein by reference in their entireties). As such, the cargo RNA may be characterized as a hybrid RNA including the RNA-motif for binding to the RNA-binding domain of the fusion protein and including an additional RNA (e.g., miRNA, shRNA, mRNA, ncRNA, sgRNA, or a combination of any of these RNAs fused at the 5′-terminus or 3′-terminus or at an internal portion within the RNA), which may be a therapeutic RNA.

In other embodiments of the disclosed extracellular vesicles, the RNA-binding domain of the fusion protein is an RNA-binding domain of the N-protein of a lambdoid bacteriophage, which may include but is not limited to lambda bacteriophage, P22 bacteriophage, and phi21 bacteriophage. (See, e.g., Keryer-Bibens et al.; Bahadur et al., “Binding of the Bacteriophage P22 N-peptide to the boxB RNA-motif Studied by Molecule Dynamics Simulations,” Biophysical J., Vol., 97, December 2009, 3139-3149; Cilley et al., “Structural mimicry in the phage phi21 N peptide-boxB RNA complex,” RNA (2003), 9:663-376; the contents of which are incorporated herein by reference in their entireties). The full-length amino acid sequence of the N-protein of lambda bacteriophage, P22 bacteriophage, and phi21 bacteriophage are provided herein as SEQ ID NOs:10, 11, and 12, respectively. The fusion proteins disclosed herein may include the full-length amino acid sequence of the N-protein of the lambdoid bacteriophage or a variant thereof as contemplated herein having a percentage of sequence identity in comparison to the amino acid sequence of the N-protein of the lambdoid bacteriophage, or a fragment thereof comprising a portion of the N-protein of the lambdoid bacteriophage (e.g., the RNA-binding domain of the N-protein of any of lambda bacteriophage, P22 bacteriophage, and phi21 bacteriophage, or SEQ ID NOs:13, 14, and 15, comprising portions of the N-proteins of lambda bacteriophage, P22 bacteriophage, and phi21 bacteriophage, respectively).

In embodiments where the fusion protein comprises an RNA-binding domain of coat protein of a lambdoid bacteriophage, the cargo RNA typically comprises an RNA-motif of lambda bacteriophage RNA which may form a high affinity binding loop called “boxB” that binds to the RNA-binding domain of the fusion protein. (See Keryer-Bibens et al.). BoxB of lambdoid bacteriophage has been characterized. (See id.; Bahadur, et al.; and Cilley et al.). For lambda bacteriophage, boxB has been determined to comprise minimally a 15-nt stem-loop structure where the identity of the nucleotides forming the stem and loop influence the affinity of the coat protein for the RNA-motif (See Keryer-Bibens et al.). In some embodiments of the disclosed extracellular vesicles, the RNA-motif comprises one or more high affinity binding loops comprising a sequence and structure selected from the group consisting of:

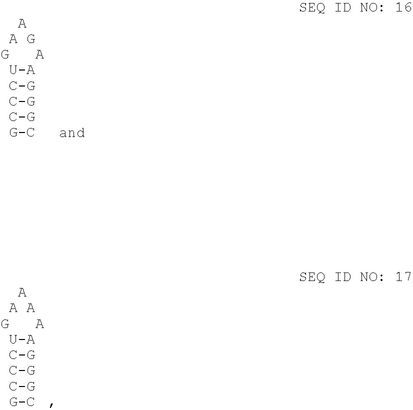

or a variant thereof having a percentage sequence identity, where the variant binds to the RNA-binding domain of the fusion protein. Preferably, the RNA-motif binds to the RNA-binding domain of the fusion protein with an affinity of at least about 1×10⁻⁸ M, more preferably with an affinity of at least about 1×10⁻⁹ M, even more preferably with an affinity of at least about 1×10⁻¹⁰ M.

For P22 bacteriophage, boxB has been determined to comprise minimally a 15-nt stem-loop structure where the identity of the nucleotides forming the stem and loop influence the affinity of the coat protein for the RNA-motif (See Bahadur et al.). In some embodiments of the disclosed extracellular vesicles, the RNA-motif comprises one or more high affinity binding loops comprising a sequence and structure of:

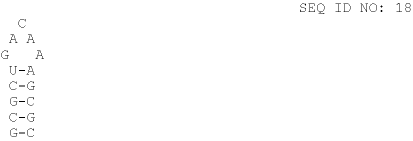

For phi21 bacteriophage, boxB has been determined to comprise minimally a 20-nt stem-loop structure where the identity of the nucleotides forming the stem and loop influence the affinity of the coat protein for the RNA-motif. (See Cilley et al.). In some embodiments of the disclosed extracellular vesicles, the RNA-motif comprises one or more high affinity binding loops comprising a sequence and structure of:

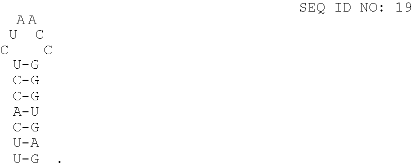

In some embodiments, the fusion protein of the disclosed extracellular vesicles comprises an RNA-binding domain of a Cas9 protein. In such embodiments, the disclosed extracellular vesicles may comprise a cargo RNA comprising a sequence that is recognized and bound by the RNA-binding domain and actively packaged into the extracellular vesicles.

The disclosed extracellular vesicles may be prepared by methods known in the art. For example, the disclosed extracellular vesicles may be prepared by expressing in a eukaryotic cell (a) an mRNA that encodes the packaging/fusion protein and (b) expressing in the eukaryotic cell the cargo RNA or cargo protein (or transducing the eukaryotic cell with the cargo RNA that has been prepared in silico). The mRNA for the packaging/fusion protein and the cargo RNA may be expressed from vectors that are transfected into suitable production cells for producing the disclosed extracellular vesicles. Note that the vector may also be stably transfected. The mRNA for the packaging/fusion protein and the cargo RNA may be expressed from the same vector (e.g., where the vector expresses the mRNA for the packaging/fusion protein and the cargo RNA from separate promoters), or the mRNA for the packaging/fusion protein and the cargo RNA may be expressed from separate vectors. The vector or vectors for expressing the mRNA for the packaging/fusion protein and the cargo RNA may be packaged in a kit designed for preparing the disclosed extracellular vesicles.

Also contemplated herein are methods for using the disclosed extracellular vesicles. For example, the disclosed extracellular vesicles may be used for delivering a therapeutic agent such as cargo RNA or cargo protein or cargo RNA-protein complexes to a target cell, where the methods include contacting the target cell with the disclosed extracellular vesicles. The disclosed extracellular vesicles may be formulated as part of a pharmaceutical composition for treating a disease or disorder and the pharmaceutical composition may be administered to a patient in need thereof to delivery the cargo molecules to target cells in order to treat the disease or disorder.

The disclosed extracellular vesicles may include a cargo protein (e.g., a therapeutic protein or a protein/RNA comples). In some embodiments, the therapeutic protein is actively packaged in the extracellular vesicles (e.g., via an interaction between the therapeutic protein and the fusion protein).

The disclosed extracellular vesicles may comprise novel proteins, polypeptides, or peptides. As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide (e.g., any of SEQ ID NOs: 1-40). The sequence of the full-length coat protein of MS2 bacteriophage, the sequence of the full-length N-protein of lambda bacteriophage, the sequence of the full-length N-protein of P22 bacteriophage, the sequence of the full-length N-protein of phi21 bacteriophage, the sequence of the full-length LAMP-2a, the sequence of the full-length LAMP-2b, and the sequence of the full-length LAMP-2c, are presented as SEQ ID NOs:1, 10, 11, 12, 20, 21, and 22, respectively, and may be used as a reference in this regard.

Regarding proteins, a “deletion” refers to a change in the amino acid sequence that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant,” “mutant,” or “derivative” of a reference polypeptide sequence may include a deletion relative to the reference polypeptide sequence.

Regarding proteins, “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide; in other embodiments, a fragment may comprise less than about 5, 10, 15, 20, 25, 30, 40, 50, 60, 70, 80, 90, 100, 150, 250, or 500 contiguous amino acid residues of a reference polypeptide; or in other embodiments, a fragment has a length within a range bounded by any of these values (e.g., a range of 50-100 contiguous amino acids of a reference polypeptide). Fragments may be preferentially selected from certain regions of a molecule. The term “at least a fragment” encompasses the full length polypeptide. For example, a fragment of a protein may comprise or consist essentially of a contiguous portion of an amino acid sequence of the full-length proteins of any of SEQ ID NOs: 1-40. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

Regarding proteins, the words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues, or a range of amino acid residues bounded by any of these values (e.g., an insertion or addition of 5-10 amino acids). A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

A “fusion polypeptide” refers to a polypeptide comprising at the N-terminus, the C-terminus, or at both termini of its amino acid sequence a heterologous amino acid sequence. A “variant” of a reference polypeptide sequence may include a fusion polypeptide comprising the reference polypeptide.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases. As described herein, variants, mutants, or fragments (e.g., a protein variant, mutant, or fragment thereof) may have 99%, 98%, 97%, 96%, 95%, 94%, 93%, 92%, 91%, 90%, 80%, 70%, 60%, 50%, 40%, 30%, or 20% amino acid sequence identity relative to a reference molecule (e.g., relative to a any of SEQ ID NOs: 1-40).

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. The following table provides a list of exemplary conservative amino acid substitutions which are contemplated herein:

| Original | |
|---|---|
| Residue | Conservative Substitute |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed proteins, mutants, variants, or described herein may have one or more functional or biological activities exhibited by a reference polypeptide (e.g., one or more functional or biological activities exhibited by wild-type protein). For example, the disclosed proteins, mutants, variants, or derivatives thereof may have one or more biological activities that include binding to a single-stranded RNA, binding to a double-stranded RNA, binding to a target polynucleotide sequence, and targeting a protein to a vesicle (e.g. a lysosome or exosome).

The disclosed proteins may be substantially isolated or purified. The term “substantially isolated or purified” refers to proteins that are removed from their natural environment, and are at least 60% free, preferably at least 75% free, and more preferably at least 90% free, even more preferably at least 95% free from other components with which they are naturally associated.

Also disclosed herein are polynucleotides, for example polynucleotide sequences that encode proteins (e.g., DNA that encodes a polypeptide having the amino acid sequence of any of any of SEQ ID NOs: 1-40 or a polypeptide variant having an amino acid sequence with at least about 20%, 30%, 40%, 50%, 60%, 70%, 80%, 90%, 95%, 96%, 97%, 98%, or 99% sequence identity to any of SEQ ID NOs: 1-40; DNA encoding the polynucleotide sequence of any of any of SEQ ID NOs: 1-40 or encoding a polynucleotide variant having a nucleotide sequence with at least about 20%, 30%, 40%, 50%, 60%, 70%, 80%, 90%, 95%, 96%, 97%, 98%, or 99% sequence identity to any of any of SEQ ID NOs: 1-40; RNA comprising the polynucleotide sequence of any of SEQ ID NOs: 1-40 or a polynucleotide variant having a nucleotide sequence with at least about 20%, 30%, 40%, 50%, 60%, 70%, 80%, 90%, 95%, 96%, 97%, 98%, or 99% sequence identity to any of SEQ ID NOs: 1-40).

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

“Transformation” or “transfected” describes a process by which exogenous nucleic acid (e.g., DNA or RNA) is introduced into a recipient cell. Transformation or transfection may occur under natural or artificial conditions according to various methods well known in the art, and may rely on any known method for the insertion of foreign nucleic acid sequences into a prokaryotic or eukaryotic host cell. The method for transformation or transfection is selected based on the type of host cell being transformed and may include, but is not limited to, bacteriophage or viral infection or non-viral delivery. Methods of non-viral delivery of nucleic acids include lipofection, nucleofection, microinjection, electroporation, heat shock, particle bombardment, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term “transformed cells” or “transfected cells” includes stably transformed or transfected cells in which the inserted DNA is capable of replication either as an autonomously replicating plasmid or as part of the host chromosome, as well as transiently transformed or transfected cells which express the inserted DNA or RNA for limited periods of time. In another embodiment, the term also includes stably transfected cells.

The polynucleotide sequences contemplated herein may be present in expression vectors. For example, the vectors may comprise: (a) a polynucleotide encoding an ORF of a protein; (b) a polynucleotide that expresses an RNA that directs RNA-mediated binding, nicking, and/or cleaving of a target DNA sequence; and both (a) and (b). The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed. For example, a heterologous promoter for a LAMP may include a eukaryotic promoter or a prokaryotic promoter that is not the native, endogenous promoter for the LAMP.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide.

Any of the conventional vectors used for expression in eukaryotic cells may be used for directly introducing DNA into a subject. Expression vectors containing regulatory elements from eukaryotic viruses may be used in eukaryotic expression vectors (e.g., vectors containing SV40, CMV, or retroviral promoters or enhancers). Exemplary vectors include those that express proteins under the direction of such promoters as the SV40 early promoter, SV40 later promoter, metallothionein promoter, human cytomegalovirus promoter, murine mammary tumor virus promoter, and Rous sarcoma virus promoter. Expression vectors as contemplated herein may include eukaryotic or prokaryotic control sequences that modulate expression of a heterologous protein (e.g. the fusion protein disclosed herein). Prokaryotic expression control sequences may include constitutive or inducible promoters (e.g., T3, T7, Lac, trp, or phoA), ribosome binding sites, or transcription terminators.

The vectors contemplated herein may be introduced and propagated in a prokaryote, which may be used to amplify copies of a vector to be introduced into a eukaryotic cell or as an intermediate vector in the production of a vector to be introduced into a eukaryotic cell (e.g. amplifying a plasmid as part of a viral vector packaging system). A prokaryote may be used to amplify copies of a vector and express one or more nucleic acids, such as to provide a source of one or more proteins for delivery to a host cell or host organism. Expression of proteins in prokaryotes may be performed using Escherichia coli with vectors containing constitutive or inducible promoters directing the expression of either a protein or a fusion protein comprising a protein or a fragment thereof. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; (iii) to aid in the purification of the recombinant protein by acting as a ligand in affinity purification (e.g., a His tag); (iv) to tag the recombinant protein for identification (e.g., such as Green fluorescence protein (GFP) or an antigen (e.g., HA) that can be recognized by a labelled antibody); (v) to promote localization of the recombinant protein to a specific area of the cell (e.g., where the protein is fused (e.g., at its N-terminus or C-terminus) to a nuclear localization signal (NLS) which may include the NLS of SV40, nucleoplasmin, C-myc, M9 domain of hnRNP A1, or a synthetic NLS). The importance of neutral and acidic amino acids in NLS have been studied. (See Makkerh et al. (1996) Curr Biol 6(8):1025-1027). Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

The presently disclosed methods may include delivering one or more polynucleotides, such as or one or more vectors as described herein, one or more transcripts thereof, and/or one or proteins transcribed therefrom, to a host cell. Further contemplated are host cells produced by such methods, and organisms (such as animals, plants, or fungi) comprising or produced from such cells. The disclosed extracellular vesicles may be prepared by introducing vectors that express mRNA encoding a fusion protein and a cargo RNA as disclosed herein. Conventional viral and non-viral based gene transfer methods can be used to introduce nucleic acids in mammalian cells or target tissues. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell.

In the methods contemplated herein, a host cell may be transiently or non-transiently transfected (i.e., stably transduced) with one or more vectors described herein. In some embodiments, a cell is transfected as it naturally occurs in a subject (i.e., in situ). In some embodiments, a cell that is transfected is taken from a subject (i.e., explanted). In some embodiments, the cell is derived from cells taken from a subject, such as a cell line. Suitable cells may include stem cells (e.g., embryonic stem cells and pluripotent stem cells). A cell transfected with one or more vectors described herein may be used to establish a new cell line comprising one or more vector-derived sequences. In the methods contemplated herein, a cell may be transiently transfected with the components of a system as described herein (such as by transient transfection of one or more vectors, or transfection with RNA), and modified through the activity of a complex, in order to establish a new cell line comprising cells containing the modification but lacking any other exogenous sequence.

### ILLUSTRATIVE EMBODIMENTS

The following embodiments are illustrative and are not intended to limit the scope of the claimed invention.

### Embodiment 1

Extracellular vesicles comprising a targeting protein, wherein the targeting protein is a fusion protein comprising: (i) a single-chain variable fragment of an antibody (scFv), wherein the scFv is expressed on the surface of the extracellular vesicles; and (ii) a transmembrane domain (TMD), wherein the scFv and TMD are directly linked or indirectly linked via a linker.

### Embodiment 2

The extracellular vesicles of embodiment 1, wherein the extracellular vesicles are exosomes or microvesicles.

### Embodiment 3

The extracellular vesicles of embodiment 1 or embodiment 2, wherein the fusion protein has a structure: Nₜₑᵣ-V_{L}-L-V_{H}-L₂-TMD-Cₜₑᵣ or Nₜₑᵣ-V_{H}-L-V_{L}-L₂-TMD-Cₜₑᵣ, wherein Nₜₑᵣ is the N-terminus, V_{L} is a variable light chain fragment of an antibody, L₁ is a first linker of about 10-50 amino acids selected from glycine, serine, and threonine, V_{H} is a variable heavy chain fragment of an antibody, L₂ is a second linker of about 10-50 amino acids optionally selected from glycine, serine, and threonine or a sequence selected from SEQ ID NOs; 41-46, TMD is a transmembrane domain, and Cₜₑᵣ is the C-terminus.

### Embodiment 4

The extracellular vesicles of any of the foregoing embodiments, further comprising an N-terminal protein tag, a C-terminal protein tag, or both of an N-terminal protein tag and a C-terminal protein tag.

### Embodiment 5

The extracellular vesicles of any of the foregoing embodiments, wherein the transmembrane targets the fusion protein to the membrane of the extracellular vesicles.

### Embodiment 6

The extracellular vesicles of any of the foregoing embodiments, wherein the transmembrane domain is a transmembrane domain of a cellular receptor protein.

### Embodiment 7

The extracellular vesicles of embodiment 6, wherein the cellular receptor protein is platelet-derived growth factor receptor.

### Embodiment 8

The extracellular vesicles of any of the foregoing embodiments, wherein the transmembrane domain is a transmembrane domain of a lysosome-associated membrane protein.

### Embodiment 9

The extracellular vesicles of any of the foregoing embodiments, wherein the lysosome membrane protein comprises a luminal N-terminal end and a cytoplasmic C-terminal end.

### Embodiment 10

The extracellular vesicles of any of the foregoing embodiments, wherein the transmembrane domain comprises the transmembrane domain of LAMP-1 or LAMP-2.

### Embodiment 11

The extracellular vesicles of any of the foregoing embodiments, wherein the fusion protein further comprises: (iii) an engineered glycosylation site.

### Embodiment 12

The extracellular vesicles of embodiment 11, wherein the fusion protein has a structure selected from: (i) Nₜₑᵣ-V_{L}-L-V_{H}-L₂-EGS-TMD-(optional RBD)-Cₜₑᵣ; (ii) Nₜₑᵣ-V_{L}-L-V_{H}-EGS-L₂-TMD-(optional RBD)-Cₜₑᵣ; (iii) Nₜₑᵣ-V_{H}-L-V_{L}-L₂-EGS-TMD-(optional RBD)-Cₜₑᵣ; and (iv) Nₜₑᵣ-V_{H}-L-V_{L}-EGS-L₂-TMD-(optional RBD)-Cₜₑᵣ; wherein Nₜₑᵣ is the N-terminus, V_{L} is a variable light chain fragment of an antibody, L₁ is a first linker of about 10-50 amino acids selected from glycine, serine, and threonine, V_{H} is a variable heavy chain fragment of an antibody, L₂ is a second linker of about 10-50 amino acids optionally selected from glycine, serine, and threonine or a sequence selected from SEQ ID NOs; 41-46, EGS is an engineered glycosylation site, TMD is a transmembrane domain, and Cₜₑᵣ is the C-terminus.

### Embodiment 13

The extracellular vesicles of embodiment 11 or 12, wherein the glycosylation site comprises a sequence selected from SEQ ID NO:37 and SEQ ID NO:38.

### Embodiment 14

The extracellular vesicles of any of the foregoing embodiments, wherein the fusion protein further comprises: (iv) an exosome-targeting domain.

### Embodiment 15

The extracellular vesicles of embodiment 14, wherein the fusion protein has a structure: (i) Nₜₑᵣ-V_{L}-L-V_{H}-L₂-ETD-TMD-(optional RBD)-Cₜₑᵣ; (ii) Nₜₑᵣ-V_{L}-L-V_{H}-L₂-TMD-ETD-(optional RBD)-Cₜₑᵣ; (iii) Nₜₑᵣ-V_{H}-L-V_{L}-L₂-ETD-TMD-(optional RBD)-Cₜₑᵣ; and (iv) Nₜₑᵣ-V_{H}-L-V_{L}-L₂-TMD-ETD-(optional RBD)-Cₜₑᵣ; wherein Nₜₑᵣ is the N-terminus, V_{L} is a variable light chain fragment of an antibody, L₁ is a first linker of about 10-50 amino acids selected from glycine, serine, and threonine, V_{H} is a variable heavy chain fragment of an antibody, L₂ is a second linker of about 10-50 amino acids optionally selected from glycine, serine, and threonine or a sequence selected from SEQ ID NOs; 41-46, TMD is a transmembrane domain, ETD is an exosome targeting domain, and Cₜₑᵣ is the C-terminus.

### Embodiment 16

The extracellular vesicles of embodiment 14 or 15, wherein the exosome-targeting domain comprises a sequence selected from a group consisting of SEQ ID NO:23, SEQ ID NO:24, SEQ ID NO:25, SEQ ID NO:27, SEQ ID NO:28, SEQ ID NO:29, SEQ ID NO:31, and SEQ ID NO:34, SEQ ID NO:35, and SEQ ID NO:36, or a variant thereof having at least 80% amino acid sequence identity to SEQ ID NO:23, SEQ ID NO:24, SEQ ID NO:25, SEQ ID NO:27, SEQ ID NO:28, SEQ ID NO:29, SEQ ID NO:31, and SEQ ID NO:34, SEQ ID NO:35, and SEQ ID NO:36, respectively.

### Embodiment 17

The extracellular vesicles of any of the foregoing embodiments, wherein the extracellular vesicles further comprise a therapeutic agent selected from the group consisting of a small molecule therapeutic, a therapeutic RNA, and a therapeutic protein.

### Embodiment 18

The extracellular vesicles of any of the foregoing embodiments, wherein the extracellular vesicles further comprise a therapeutic RNA as a cargo RNA and the fusion protein further comprises an RNA-binding domain for the cargo RNA, and/or the extracellular vesicles further comprise a therapeutic protein as a cargo protein and the fusion protein further comprises a domain that binds to a cognate domain on the therapeutic protein.

### Embodiment 19

The extracellular vesicles of embodiment 18, wherein the fusion protein has a structure: Nₜₑᵣ-V_{L}-L₁-V_{H}-TMD-RBD-Cₜₑᵣ or Nₜₑᵣ-V_{H}-L1-V_{L}-TMD-RBD-Cₜₑᵣ, wherein Nₜₑᵣ is the N-terminus, V_{L} is a variable light chain fragment of an antibody, L₁ is a linker of about 10-60 amino acids selected from glycine, serine, and threonine, V_{H} is a variable heavy chain fragment of an antibody, TMD is a transmembrane domain, RBD is the RNA-binding domain for the cargo RNA, and Cₜₑᵣ is the C-terminus.

### Embodiment 20

The extracellular vesicles of embodiment 18, wherein the cargo RNA comprises an RNA-motif and the RNA-binding domain of the fusion protein binds specifically to the RNA-motif of the cargo RNA.

### Embodiment 21

The extracellular vesicles of embodiment 18, wherein the RNA-binding domain is an RNA-binding domain of a bacteriophage, and wherein the RNA-motif comprises one or more high affinity binding loops of RNA of the bacteriophage.

### Embodiment 22

The extracellular vesicles of embodiment 21, wherein the RNA-binding domain is the RNA-binding domain of MS2 bacteriophage comprising SEQ ID NO:2 or a variant thereof having at least 80% amino acid sequence identity to SEQ ID NO:2, and wherein the RNA-motif comprises one or more high affinity binding loops comprising a sequence and structure selected from the group consisting of:

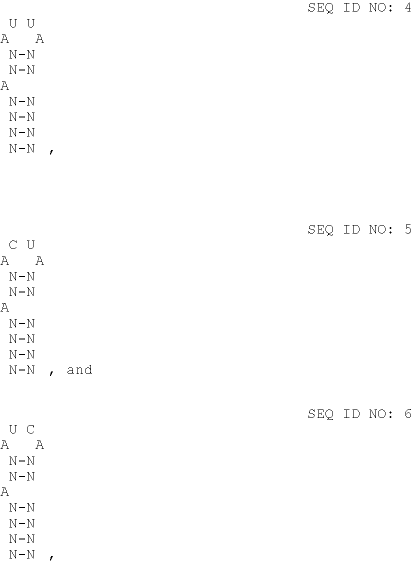

where N—N is any two base-paired RNA nucleotides.

### Embodiment 23

The extracellular vesicles of embodiment 21, wherein the high affinity binding loop comprises a sequence selected from the group consisting of SEQ ID NO:7, SEQ ID NO:8, and SEQ ID NO:9, or a variant thereof having at least 80% amino acid sequence identity to SEQ ID NO:7, SEQ ID NO:8, and SEQ ID NO:9, respectively.

### Embodiment 24

The extracellular vesicles of embodiment 23, wherein the RNA-binding domain is the RNA-binding domain of the N-protein of lambda bacteriophage comprising SEQ ID NO:13 or a variant thereof having at least 80% amino acid sequence identity to SEQ ID NO:13, and wherein the RNA-motif comprises one or more high affinity binding loops comprising a sequence and structure selected from the group consisting of:

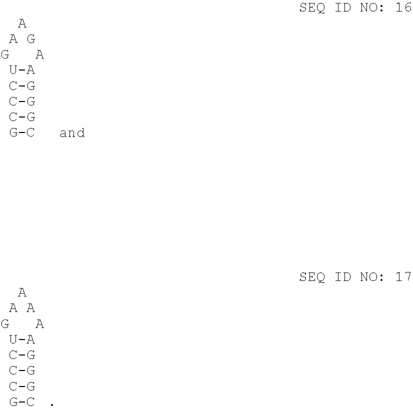

Embodiment 25

The extracellular vesicles of embodiment 21, wherein the RNA-binding domain is the RNA-binding domain of the N-protein of P22 bacteriophage comprising SEQ ID NO:14 or a variant thereof having at least 80% amino acid sequence identity to SEQ ID NO:14, and wherein the RNA-motif comprises one or more high affinity binding loops comprising a sequence and structure of:

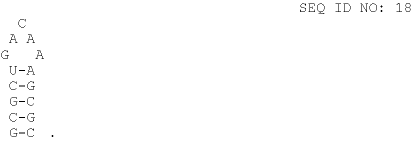

Embodiment 26

The extracellular vesicles of embodiment 25, wherein the RNA-binding domain is the RNA-binding domain of the N-protein of phi22 bacteriophage comprising SEQ ID NO:15 or a variant thereof having at least 80% amino acid sequence identity to SEQ ID NO:15, and wherein the RNA-motif comprises one or more high affinity binding loops comprising a sequence and structure of:

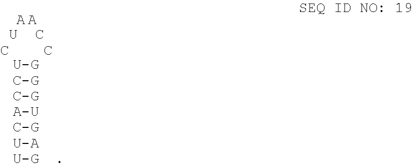

Embodiment 27

The extracellular vesicles of embodiment 18, wherein the cargo RNA is a hybrid RNA comprising the RNA-motif and further comprising miRNA, shRNA, mRNA, ncRNA, sgRNA, or a combination of any of these RNAs.

### Embodiment 28

A method for preparing the extracellular vesicles of any of the foregoing embodiment, the method comprising expressing in a eukaryotic cell an mRNA that encodes the fusion protein.

### Embodiment 29

A method for preparing the extracellular vesicles of embodiment 18, the method comprising: (a) expressing in a eukaryotic cell an mRNA that encodes the fusion protein and (b) expressing in a eukaryotic cell the cargo RNA or transducing the eukaryotic cell with the cargo RNA, or expressing the cargo protein.

### Embodiment 30

A kit for preparing the extracellular vesicles of embodiment 18, the kit comprising: (a) a vector for expressing the fusion protein, and (b) a vector for expressing the cargo RNA or the cargo protein or RNA/protein complex.

### Embodiment 31

The kit of embodiment 30, wherein the vectors are separate vectors.

### EXAMPLES

The following Examples are illustrative and are not intended to limit the scope of the claimed subject matter.

### Example 1

Reference is made to the poster presentation entitled “Engineered extracellular vesicle-mediated delivery of targeted nucleases to inactivate HIV proviral DNA,” Devin M. Stranford and Joshua N. Leonard, presented on Oct. 2, 2017, at the Third Coast Center for AIDS Research (CFAR) Symposium, the content of which is incorporated herein by reference in its entirety.

Engineered Extracellular Vesicle-Mediated Delivery of Targeted Nucleases to Inactivate HIV Proviral DNA

Introduction

A major barrier to curing HIV infection is the persistence of a latent viral reservoir in cells. Recently it has been demonstrated that the use of Cas9 and combinatorial guide RNAs can damage latent proviruses and prevent viral escape. This pilot project will investigate the use of extracellular vesicles to deliver Cas9 therapies to T cells in a clinically translatable manner

Opportunity

Latent HIV proviruses contribute to viral load upon treatment interruption or failure, and eliminating such reservoirs is an unmet clinical need. A promising strategy is the use of engineered nucleases, such as Cas9, targeting the HIV genome in T cells to damage proviral DNA. While such approached impair viral replications in vitro, translating this approach requires overcoming several challenges.

Challenges

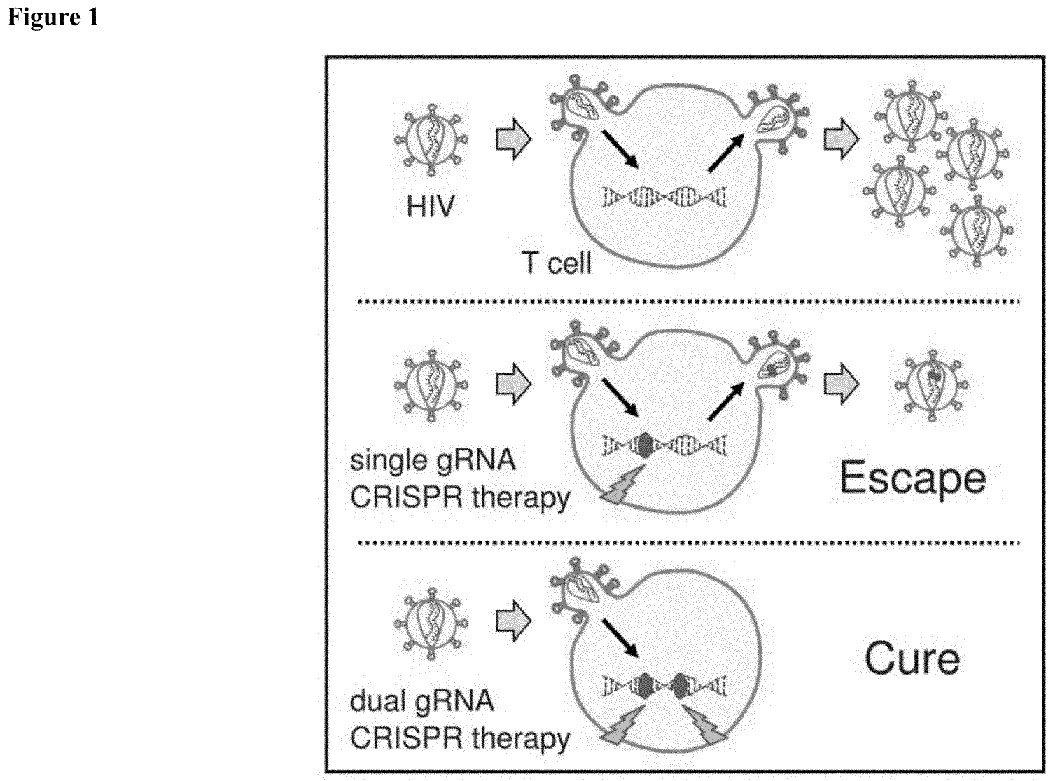

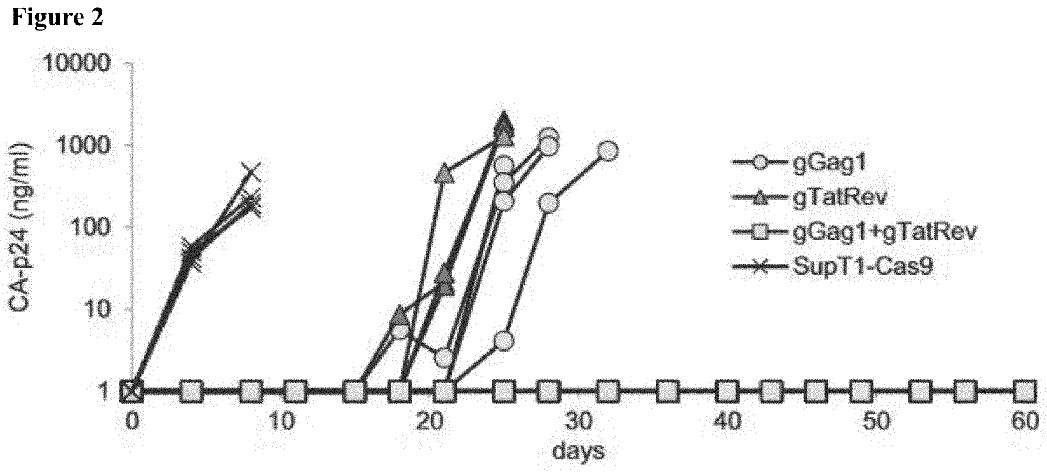

HIV rapidly escapes from nucleases targeted at protein-coding or non-essential sequences. (See FIG. 1). However, a recent report demonstrated that simultaneously targeting certain pairs of HIV loci with Cas9 suppressed viral replication and escape. (See FIG. 2, from Wang et al. “A Combinatorial CRISPR-Cas9 Attack on HIV-1 DNA Extinguishes All Infectious Provirus in Infected T Cell Cultures, Cell Reports, Volume 17, Issue 11, p2819-2826, Dec. 13, 2016; the content of which is incorporated herein by reference in its entirety). In practice, elimination of virus may require multiplexed and perhaps sequential targeted nuclease treatments to suppress emergent viruses.

Additionally, no readily translatable strategy for delivering nucleases to Tcells has been identified, particularly if multiple rounds/types of treatment are required. Therefore, new methods for delivering targeted therapeutics to Tcells invivo are required.

Strategy

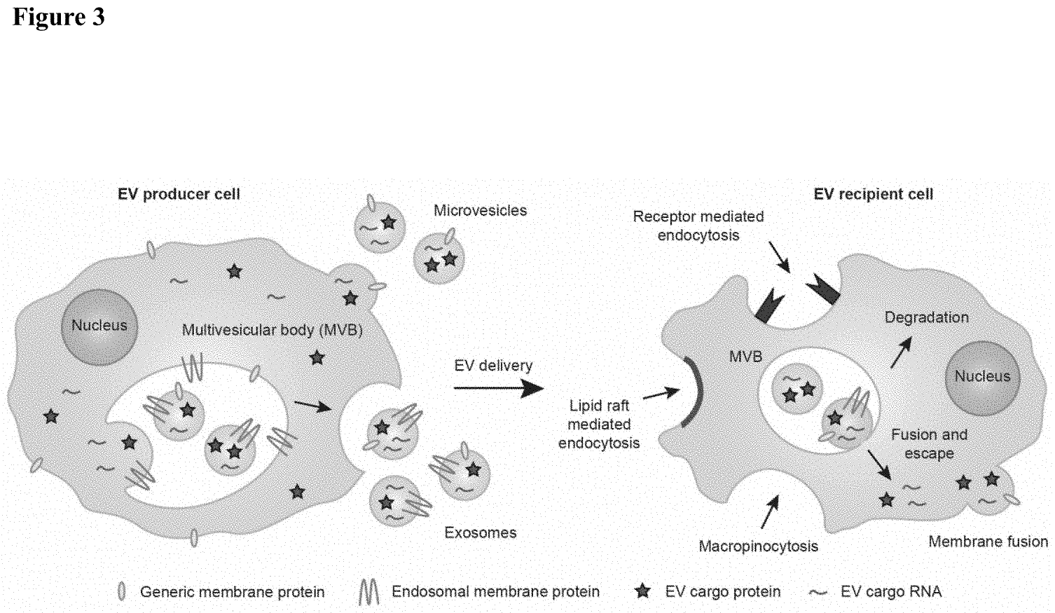

EVs are nanoscale particles that transfer RNA and proteins between many types of cells. (See FIG. 3). Increasingly, EVs are considered to be viable therapeutic delivery vehicles, since they exhibit favorable stability, non-toxicity, and delivery compared to synthetic delivery vehicles. The ability to engineer EVs to load desired cargo and target certain cells makes them promising vehicles for nuclease delivery to T cells.

Goals

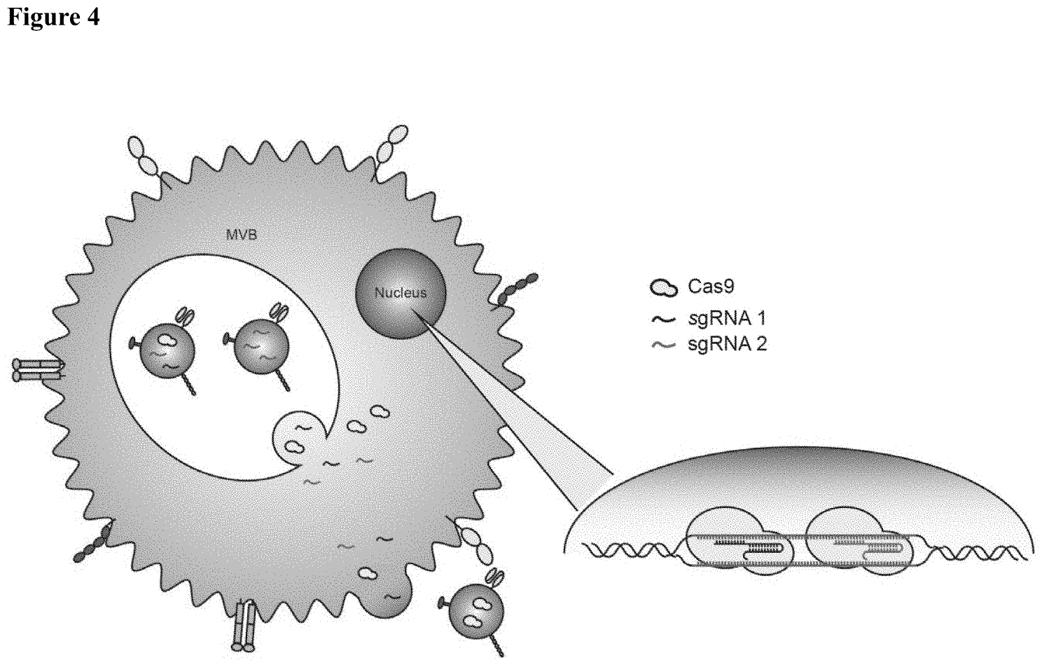

We aim to develop a novel strategy for delivering therapeutic biomolecules to T cells by harnessing secreted EV-mediated transfer. Specifically, we will explore different methods for targeting EVs to T cells by displaying various proteins on the EV surface and investigate loading and delivery of Cas9 protein or mRNA in combination with multiple guideRNAs. (See FIG. 4).

Methods of Engineering EVs to Target T Cells

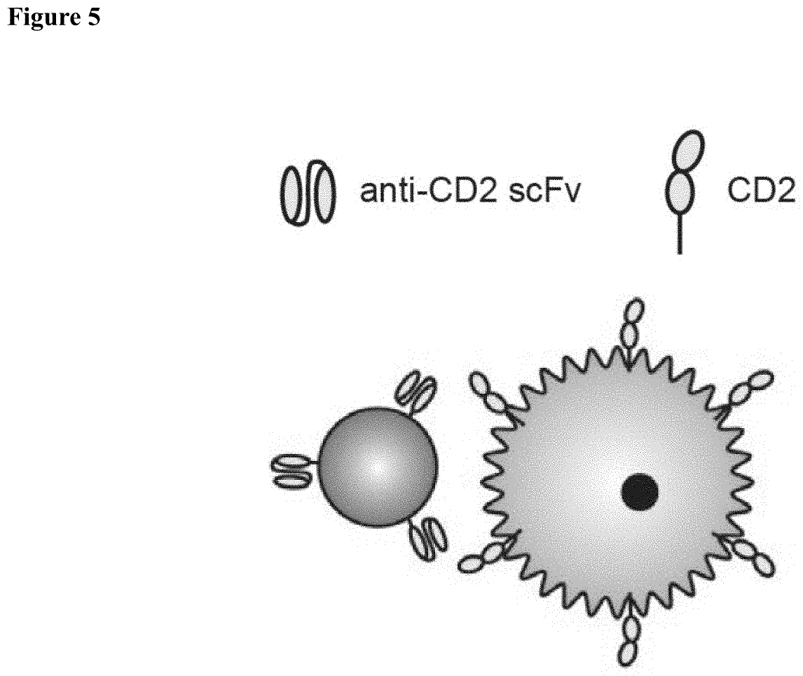

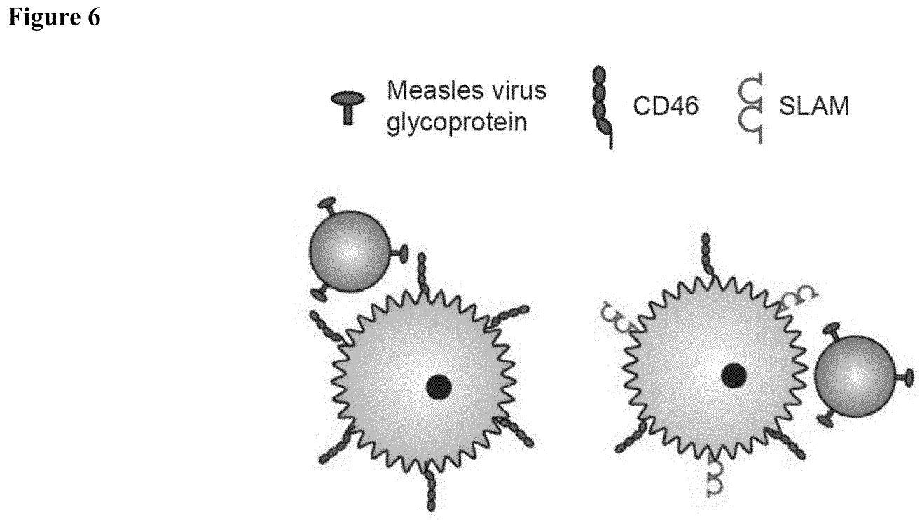

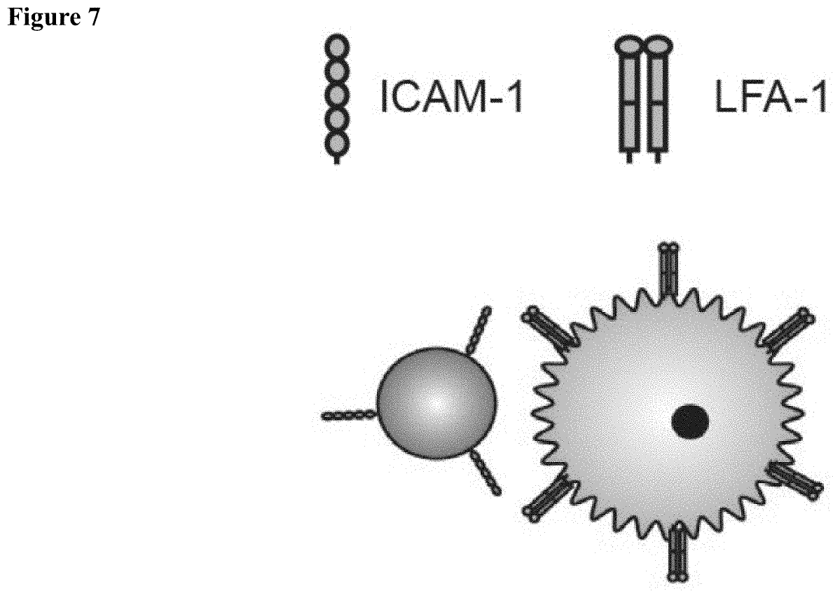

Overproducing cargo of interest in EV producer cells leads to increased accumulation in EVs. Producer HEK293FT cells will be transfected with various T cell targeting constructs to created EVs displaying such constructs. FIG. 5 illustrates EVs displaying anti-CD2 scFV which targets these EVs to CD2-bearing cells such as T cells that are latently infected with HIV. FIG. 6 illustrates EVs displaying measles virus glycoprotein variants H and F which targets these EVs to CD46-bearing cells and Signalling Lymphocyte Activation Molecule (SLAM)-bearing cells. These EVs can be utilized to transduce resting T cells. FIG. 7 illustrates EVs displaying intercellular Adhesion Molecule 1 (ICAM-1) which targets these EVs to Lymphocyte Function-Associated Antigen 1 (LFA-1)-bearing cells. These EVs can be utilized to increase uptake of dendritic cell-derived EVs.

Methods of Loading EVs with Cas9 and sgRNA

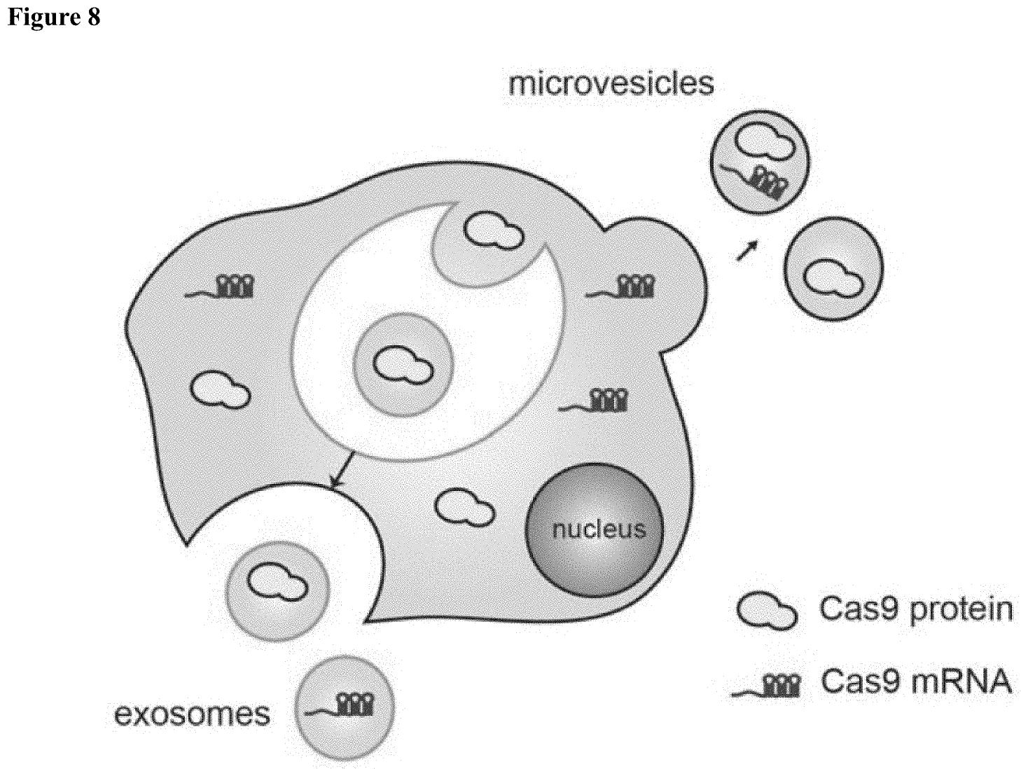

Producer cells will be transfected with Cas9 and sgRNAs to investigate loading and functional delivery to recipient cells. (See FIG. 8). Engineered interactions between Cas9 protein or mRNA and EV-enriched proteins will be explored to increase loading if needed.

scFV Display on EVs

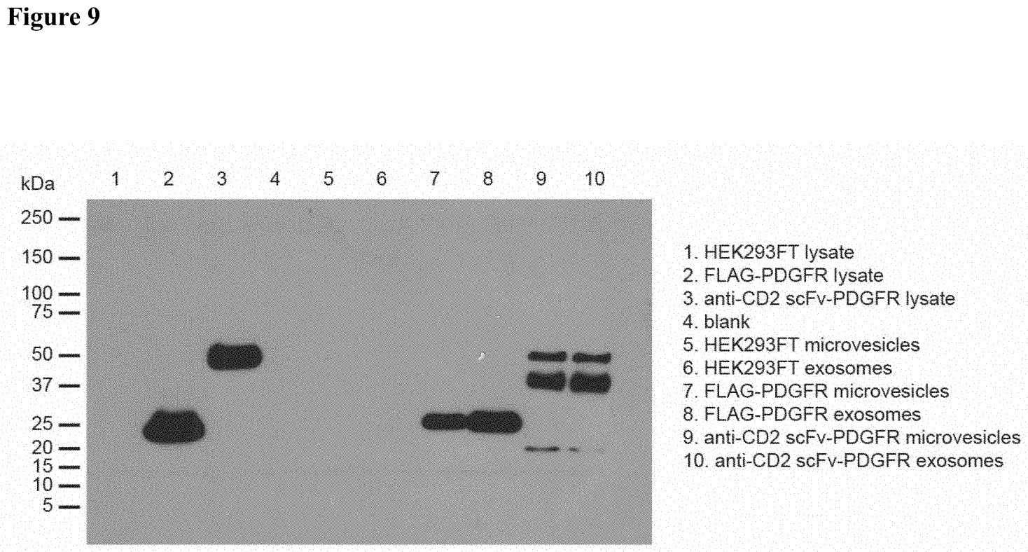

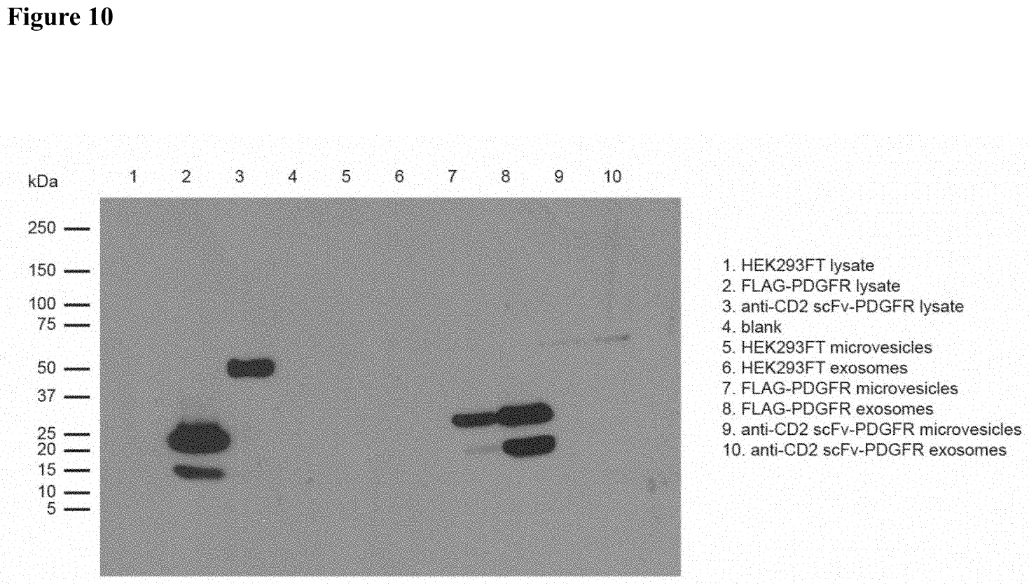

Need: Because T cells exhibit low rates of endocytosis, methods are needed to increase EV uptake by recipient cells. One currently unexplored approach is to display an scFv on the surface of EVs to increase the binding between the EV and the target cell. Here, we investigated display of an anti-CD2 scFv to EVs to specifically target T cells. (See FIGS. 9 and 10).

Fusion of an anti-CD2 scFv to the platelet derived growth factor receptor transmembrane domain leads to scFv localization to two subsets of EV: microvesicles (which bud directly from the cell surface) and exosomes (which originate in the endosomal pathway).

Cell lysates (2 μg) or EVs (8.9×10⁸ per lane) were loaded and constructs were detected by anti-FLAG antibodies. (See FIGS. 9 and 10). Predicted of full length scFv construct: ˜40 kDa. FLAG-GDGFR constructs (˜12 kDa) lack the scFv region as an Ev-display control. We observed that scFvs can be displayed on multiple EV subsets.

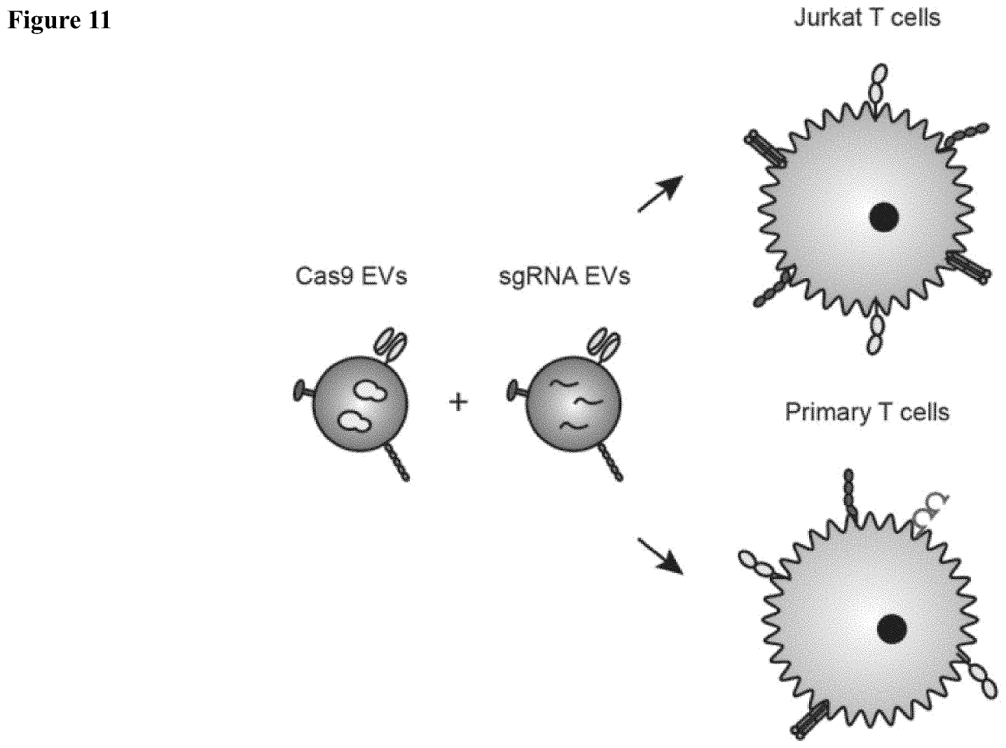

As part of ongoing work, we are exploring methods for increasing the display of scFvs on EVs. We also are investigating binding and uptake of scFv-displaying EVs to Jurkat and primarty T cells. In addition, we are displaying measles virus glycoprotein variants H and F on the surface of EVs and investigating the effect on EV uptake. Finally, we plan on evaluating the loading of Cas9 and sgRNA into EVs and functional delivery to recipient cells. (See FIG. 11).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. Extracellular vesicles comprising a targeting protein, wherein the targeting protein is a fusion protein comprising:
(i) an affinity agent wherein the affinity agent is expressed on the surface of the extracellular vesicles; and
(ii) a transmembrane domain (TMD), wherein the affinity agent and TMD are directly linked or indirectly linked via a linker.

2. The extracellular vesicles of claim 1, wherein the affinity agent is a single chain variable fragment of an antibody (scFv).

3. The extracellular vesicles of claim 2, wherein the fusion protein has a structure: Nₜₑᵣ-V_{L}-L-V_{H}-L₂-TMD-Cₜₑᵣ or Nₜₑᵣ-V_{H}-L-V_{L}-L₂-TMD-Cₜₑᵣ, wherein Nₜₑᵣ is the N-terminus, V_{L} is a variable light chain fragment of an antibody, L₁ is a first linker of about 10-50 amino acids selected from glycine, serine, and threonine, V_{H} is a variable heavy chain fragment of an antibody, L₂ is a second linker of about 10-50 amino acids optionally selected from glycine, serine, and threonine or a sequence selected from SEQ ID NOs; 41-46, TMD is a transmembrane domain, and Cₜₑᵣ is the C-terminus.

4. The extracellular vesicles of claim 1, further comprising an N-terminal protein tag, a C-terminal protein tag, or both of an N-terminal protein tag and a C-terminal protein tag.

5. The extracellular vesicles of claim 1, wherein the transmembrane targets the fusion protein to the membrane of the extracellular vesicles.

6. The extracellular vesicles of claim 1, wherein the transmembrane domain is a transmembrane domain of a cellular receptor protein.

7. The extracellular vesicles of claim 6, wherein the cellular receptor protein is platelet-derived growth factor receptor.

8. The extracellular vesicles of claim 1, wherein the transmembrane domain is a transmembrane domain of a lysosome-associated membrane protein.

9. The extracellular vesicles of claim 1, wherein the lysosome membrane protein comprises a luminal N-terminal end and a cytoplasmic C-terminal end.

10. The extracellular vesicles of claim 1, wherein the transmembrane domain comprises the transmembrane domain of LAMP-1 or LAMP-2.

11. The extracellular vesicles of claim 2, wherein the fusion protein further comprises: (iii) an engineered glycosylation site.

12. The extracellular vesicles of claim 11, wherein the fusion protein has a structure selected from:
Nₜₑᵣ-V_{L}-L-V_{H}-L₂-EGS-TMD-(optional RBD)-Cₜₑᵣ;
Nₜₑᵣ-V_{L}-L-V_{H}-EGS-L₂-TMD-(optional RBD)-Cₜₑᵣ;
Nₜₑᵣ-V_{H}-L-V_{L}-L₂-EGS-TMD-(optional RBD)-Cₜₑᵣ; and
Nₜₑᵣ-V_{H}-L-V_{L}-EGS-L₂-TMD-(optional RBD)-Cₜₑᵣ;
wherein Nₜₑᵣ is the N-terminus, V_{L} is a variable light chain fragment of an antibody, L₁ is a first linker of about 10-50 amino acids selected from glycine, serine, and threonine, V_{H} is a variable heavy chain fragment of an antibody, L₂ is a second linker of about 10-50 amino acids optionally selected from glycine, serine, and threonine or a sequence selected from SEQ ID NOs; 41-46, EGS is an engineered glycosylation site, TMD is a transmembrane domain, and Cₜₑᵣ is the C-terminus.

13. The extracellular vesicles of claim 11, wherein the glycosylation site comprises a sequence selected from SEQ ID NO:37 and SEQ ID NO:38.

14. The extracellular vesicles of claim 2, wherein the fusion protein further comprises: (iv) an exosome-targeting domain.

15. The extracellular vesicles of claim 14, wherein the fusion protein has a structure:
Nₜₑᵣ-V_{L}-L-V_{H}-L₂-ETD-TMD-(optional RBD)-Cₜₑᵣ;
Nₜₑᵣ-V_{L}-L-V_{H}-L₂-TMD-ETD-(optional RBD)-Cₜₑᵣ;
Nₜₑᵣ-V_{H}-L-V_{L}-L₂-ETD-TMD-(optional RBD)-Cₜₑᵣ; and
Nₜₑᵣ-V_{H}-L-V_{L}-L₂-TMD-ETD-(optional RBD)-Cₜₑᵣ;
wherein Nₜₑᵣ is the N-terminus, V_{L} is a variable light chain fragment of an antibody, L₁ is a first linker of about 10-50 amino acids selected from glycine, serine, and threonine, V_{H} is a variable heavy chain fragment of an antibody, L₂ is a second linker of about 10-50 amino acids optionally selected from glycine, serine, and threonine or a sequence selected from SEQ ID NOs; 41-46, TMD is a transmembrane domain, ETD is an exosome targeting domain, and Cₜₑᵣ is the C-terminus.

16. The extracellular vesicles of claim 14, wherein the exosome-targeting domain comprises a sequence selected from a group consisting of SEQ ID NO:23, SEQ ID NO:24, SEQ ID NO:25, SEQ ID NO:27, SEQ ID NO:28, SEQ ID NO:29, SEQ ID NO:31, and SEQ ID NO:34, SEQ ID NO:35, and SEQ ID NO:36, or a variant thereof having at least 80% amino acid sequence identity to SEQ ID NO:23, SEQ ID NO:24, SEQ ID NO:25, SEQ ID NO:27, SEQ ID NO:28, SEQ ID NO:29, SEQ ID NO:31, and SEQ ID NO:34, SEQ ID NO:35, and SEQ ID NO:36, respectively.

17. The extracellular vesicles of claim 1, wherein the extracellular vesicles further comprise a therapeutic agent selected from the group consisting of a small molecule therapeutic, a therapeutic RNA, and a therapeutic protein or a combination.

18. The extracellular vesicles of claim 1, wherein the extracellular vesicles further comprise a therapeutic RNA as a cargo RNA and the fusion protein further comprises an RNA-binding domain for the cargo RNA, and/or the extracellular vesicles further comprise a therapeutic protein as a cargo protein and the fusion protein further comprises a domain that binds to a cognate domain on the therapeutic protein.

19. The extracellular vesicles of claim 18, wherein the fusion protein has a structure: Nₜₑᵣ-V_{L}-L₁-V_{H}-TMD-RBD-Cₜₑᵣ or Nₜₑᵣ-V_{H}-L₁-V_{L}-TMD-RBD-Cₜₑᵣ, wherein Nₜₑᵣ is the N-terminus, V_{L} is a variable light chain fragment of an antibody, L₁ is a linker of about 10-60 amino acids selected from glycine, serine, and threonine, V_{H} is a variable heavy chain fragment of an antibody, TMD is a transmembrane domain, RBD is the RNA-binding domain for the cargo RNA, and Cₜₑᵣ is the C-terminus.

20. The extracellular vesicles of claim 18, wherein the cargo RNA is a hybrid RNA comprising the RNA-motif and further comprising miRNA, shRNA, mRNA, ncRNA, sgRNA, or a combination of any of these RNAs.

21. A method for preparing the extracellular vesicles of claim 1, the method comprising expressing in a eukaryotic cell an mRNA that encodes the fusion protein.

22. A method for preparing the extracellular vesicles of claim 18, the method comprising: (a) expressing in a eukaryotic cell an mRNA that encodes the fusion protein and (b) expressing in a eukaryotic cell the cargo RNA or transducing the eukaryotic cell with the cargo RNA, or expressing the cargo protein or both.

23. A kit for preparing the extracellular vesicles of claim 18, the kit comprising: (a) a vector for expressing the fusion protein, and (b) a vector for expressing the cargo RNA or the cargo protein.

24. The kit of claim 23, wherein the vectors are separate vectors.
